SEQUENCE LISTING (13 sequences)

SEQ ID NO: 1 - protein, 491 aa, Plasmodium falciparum
  SITE (1)..(491): GLURP amino acids 24-514
KTNTSENRNKRIGGPKLRGNVTSNIKFPSDNKGKIIRGSNDKLNKNSEDVLEQSEKSLVSENVPSGLDIDDIPKESIFIQEDQEGQTHSELNPETSEHSKDLNNNDSKNESSDIISVNNKSNKVQNHFESLSDLELLENSSQDNLDKDTISTEPFPNQKHKDLQQDLNDEPLEPFPTQIHKDYKEKNLINEEDSEPFPRQKHKKVDNHNEEKNVFHENGSANGNQGSLKLKSFDEHLKDEKIENEPLVHENLSIPNDPIEQILNQPEQETNIQEQLYNEKQNVEEKQNSQIPSLDLKEPTNEDILPNHNPLENIKQSESEINHVQDHALPKENIIDKLDNQKEHIDQSQHNINVLQENNINNHQLEPQEKPNIESFEPKNIDSEIILPENVETEEIIDDVPSPKHSNHETFEEETSESEHEEAVSEKNAHETVEHEETVSQESNPEKADNDGNVSQNSNNELNENEFVESEKSEHEPAENEESSLEEGHHE

SEQ ID NO: 2 - protein, 169 aa, Plasmodium falciparum
  SITE (1)..(169): MSP3 amino acids 212-380
KEASSYDYILGWEFGGGVPEHKKEENMLSHLYVSSKDKENISKENDDVLDEKEEEAEETEEEELEEKNEEETESEISEDEEEEEEEEKEEENEKKKEQEKEQSNENNDQKKDMEAQNLISKNQNNNEKNVKEAAESIMKTLAGLIKGNNQIDSTLKDLVEELSKYFKNH

SEQ ID NO: 3 - protein, 647 aa, Artificial Sequence
  Description of Artificial Sequence Synthetic Peptide
  SITE (1)..(647): GLURP MSP3 fusion protein
TSENRNKRIGGPKLRGNVTSNIKFPSDNKGKIIRGSNDKLNKNSEDVLEQSEKSLVSENVPSGLDIDDIPKESIFIQEDQEGQTHSELNPETSEHSKDLNNNGSKNESSDIISENNKSNKVQNHFESLSDLELLENSSQDNLDKDTISTEPFPNQKHKDLQQDLNDEPLEPFPTQIHKDYKEKNLINEEDSEPFPRQKHKKVDNHNEEKNVFHENGSANGNQGSLKLKSFDEHLKDEKIENEPLVHENLSIPNDPIEQILNQPEQETNIQEQLYNEKQNVEEKQNSQIPSLDLKEPTNEDILPNHNPLENIKQSESEINHVQDHALPKENIIDKLDNQKEHIDQSQHNINVLQENNINNHQLEPQEKPNIESFEPKNIDSEIILPENVETEEIIDDVPSPKHSNHETFEEETSESEHEEAVSEKNAHETVEHEETVSQESNPEKADNDGNVSQNSNNELNENEFVESEKSEHEARSKAKEASSYDYILGWEFGGGVPEHKKEENMLSHLYVSSKDKENISKENDDVLDEKEEEAEETEEEELEEKNEEETESEISEDEEEEEEEEKEEENEKKKEQEKEQSNENNDQKKDMEAQNLISKNQNNNEKNVKEAAESIMKTLAGLIKGNNQIDSTLKDLVEELSKYFKNH

SEQ ID NO: 4 - DNA, 1941 bp, Artificial Sequence
  Description of Artificial Sequence Synthetic DNA
acaagtgagaatagaaataaacgaatcgggggtcctaaattaaggggtaatgttacaagtaatataaagttcccatcagataacaaaggtaaaattataagaggttcgaatgataaacttaataaaaactctgaagatgttttagaacaaagcgaaaaatcgcttgtttcagaaaatgttcctagtggattagatatagatgatatccctaaagaatctatttttattcaagaagatcaagaaggtcaaactcattctgaattaaatcctgaaacatcagaacatagtaaagatttaaataataatggttcaaaaaatgaatctagtgatattatttcagaaaataataaatcaaataaagtacaaaatcattttgaatcattatcagatttagaattacttgaaaattcctcacaagataatttagacaaagatacaatttcaacagaaccttttcctaatcaaaaacataaagacttacaacaagatttaaatgatgaacctttagaaccctttcctacacaaatacataaagattataaagaaaaaaatttaataaatgaagaagattcagaaccatttcccagacaaaagcataaaaaggtagacaatcataatgaagaaaaaaacgtatttcatgaaaatggttctgcaaatggtaatcaaggaagtttgaaacttaaatcattcgatgaacatttaaaagatgaaaaaatagaaaatgaaccacttgttcatgaaaatttatccataccaaatgatccaatagaacaaatattaaatcaacctgaacaagaaacaaatatccaggaacaattgtataatgaaaaacaaaatgttgaagaaaaacaaaattctcaaataccttcgttagatttaaaagaaccaacaaatgaagatattttaccaaatcataatccattagaaaatataaaacaaagtgaatcagaaataaatcatgtacaagatcatgcgctaccaaaagagaatataatagacaaacttgataatcaaaaagaacacatcgatcaatcacaacataatataaatgtattacaagaaaataacataaacaatcaccaattagaacctcaagagaaacctaatattgaatcgtttgaacctaaaaatatagattcagaaattattcttcctgaaaatgttgaaacagaagaaataatagatgatgtgccttcccctaaacattctaaccatgaaacatttgaagaagaaacaagtgaatctgaacatgaagaagccgtatctgaaaaaaatgcccacgaaactgtcgaacatgaagaaactgtgtctcaagaaagcaatcctgaaaaagctgataatgatggaaatgtatctcaaaacagcaacaacgaattaaatgaaaatgaattcgttgaatcggaaaaaagcgagcatgaagcaagatctaaagcaaaagaagcttctagttatgattatattttaggttgggaatttggaggaggcgttccagaacacaaaaaagaagaaaatatgttatcacatttatatgtttcttcaaaggataaggaaaatatatctaaggaaaatgatgatgtattagatgagaaggaagaagaggcagaagaaacagaagaagaagaacttgaagaaaaaaatgaagaagaaacagaatcagaaataagtgaagatgaagaagaagaagaagaagaagaaaaggaagaagaaaatgaaaaaaaaaaagaacaagaaaaagaacaaagtaatgaaaataatgatcaaaaaaaagatatggaagcacagaatttaatttctaaaaaccagaataataatgagaaaaacgtaaaagaagctgctgaaagcatcatgaaaactttagctggtttaatcaagggaaataatcaaatagattctaccttaaaagatttagtagaagaattatccaaatattttaaaaatcat

SEQ ID NO: 5 - protein, 27 aa, Plasmodium falciparum
  SITE (1)..(27): MSP3b
AKEASSYDYILGWEFGGGVPEHKKEEN

SEQ ID NO: 6 - protein, 41 aa, Plasmodium falciparum
  SITE (1)..(41): MSP3d
MLSHLYVSSKDKENISKENDDVLDEKEEEAEETEEEELEEK

SEQ ID NO: 7 - protein, 188 aa, Plasmodium falciparum
  SITE (1)..(188): MSP3a to MSP3f
YEKAKNAYQKANQAVLKAKEASSYDYILGWEFGGGVPEHKKEENMLSHLYVSSKDKENISKENDDVLDEKEEEAEETEEEELEEKNEEETESEISEDEEEEEEEEEKEEENDKKKEQEKEQSNENNDQKKDMEAQNLISKNQNNNEKNVKEAAESIMKTLAGLIKGNNQIDSTLKDLVEELSKYFKNH

SEQ ID NO: 8 - DNA, 31 bp, Artificial Sequence
  Description of Artificial Sequence Synthetic Primer
cccagatctacaagtgagaatagaaataaac

SEQ ID NO: 9 - DNA, 34 bp, Artificial Sequence
  Description of Artificial Sequence Synthetic Primer
cccagatcttgcttcatgctcgcttttttccgat

SEQ ID NO: 10 - DNA, 38 bp, Artificial Sequence
  Description of Artificial Sequence Synthetic Primer
aagtagatctactaatacaagtgagaatagaaataaac

SEQ ID NO: 11 - DNA, 32 bp, Artificial Sequence
  Description of Artificial Sequence Synthetic Primer
gttcagatctttattcatgatggccttctagc

SEQ ID NO: 12 - DNA, 33 bp, Artificial Sequence
  Description of Artificial Sequence Synthetic Primer
cccagatctaaagcaaaagaagcttctagttat

SEQ ID NO: 13 - DNA, 38 bp, Artificial Sequence
  Description of Artificial Sequence Synthetic Primer
attagatctcatttaatgatttttaaaatatttggata

(19) United States
(12) Patent Application Publication
(10) Pub. No.: US 2007/0003562 A1
(43) Pub. Date: Jan. 4, 2007

(54) GLURP-MSP3 fusion protein, immunogenic compositions and malarial vaccines containing it

(75) Inventor: Pierre Druilhe, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Institut Pasteur

(21) Appl. No.: 11/389,335

(22) Filed: Mar. 27, 2006

Related U.S. Application Data
(63) Continuation of application No. 10/691,672, filed on Oct. 24, 2003, now abandoned.

(51) Int. Cl. A61K 39/00; C07K 14/445

(52) U.S. Cl. 424/184.1; 530/350

(57) ABSTRACT

The present invention relates to the protection against malaria. More particularly, the invention pertains to a chimeric molecule comprising at least one moiety from the Glutamate-rich protein (GLURP) and one second moiety from the Merozoite surface protein 3 (MSP3) of Plasmodium falciparum, wherein said chimeric molecule is able to induce an immunogenic response against both of said antigens (GLURP and MSP3), when it is administrated to an appropriate host. The present invention thus also pertains to a vaccine against malaria, comprising such a molecule.

The present invention relates to the protection against malaria. More particularly, the invention pertains to a chimeric molecule comprising at least one moiety from the Glutamate-rich protein (GLURP) and one second moiety from the Merozoite surface protein 3 (MSP3) of Plasmodium falciparum, wherein said chimeric molecule is able to induce an immunogenic response against both of said antigens (GLURP and MSP3), when it is administrated to an appropriate host. The present invention thus also pertains to a vaccine against malaria, comprising such a molecule.

Antibodies have been repeatedly shown to play an important role in the development of clinical immunity to Plasmodium falciparum malaria. Numerous immunological studies now suggest that human antibodies of the cytophilic subclasses (IgG1 and IgG3) are particularly critical to the state of premunition. This anti-parasite immunity is a strain-independent, non-sterilizing type of immunity which is acquired after lengthy exposure (15-20 years) to the parasite. It is commonly observed in Africa and in Papua-New Guinea but it has only recently been documented in S-E Asia (Soe, Khin Saw et al. 2001). Although antibodies can act directly upon merozoite invasion of red blood cells, the most efficient in vivo mechanism for antibody-mediated parasite control in endemic areas requires the participation of monocytes (Khusmith and Druilhe 1983), (Lunel and Druilhe 1989). The antibody-dependent cellular inhibition (ADCI) assay mimics this cooperation between monocytes and cytophilic parasite-specific antibodies and appears today as the best in vitro surrogate marker of acquired immunity against P. falciparum blood stages.

Two molecules have so far been identified as targets of ADCI-effective human antibodies, namely the 48-kDa Merozoite surface-protein 3,—hereafter designated as MSP3—(Oeuvray, Bouharoun-Tayoun et al. 1994) and the 220-kDa Glutamate-rich protein,—hereafter designated as GLURP—(Theisen, Soe et al. 1998). It has also been shown that GLURP and MSP3 can inhibit parasite growth in vivo by passive transfer in P. falciparum-humanized SCID mice (Badell, Oeuvray et al. 2000). The association of human antibodies against these antigens with clinical protection is also indicated by a number of immuno-epidemiological studies, which demonstrate that the levels of GLURP and MSP3 specific cytophilic antibodies (IgG1 and IgG3) are significantly associated with a reduced risk of malaria attacks (Dodoo, Theisen et al. 2000; Oeuvray, Theisen et al. 2000; Theisen, Dodoo et al. 2001). These studies have further shown that cytophilic IgG3 antibodies play a major role in protection against malaria, hence bringing epidemiological support to the concept that antibodies against GLURP and MSP3 can actively control parasite multiplication in vivo by cooperation with cells bearing FcγII receptors (Bouharoun-Tayoun, Oeuvray et al. 1995). These receptors display higher affinity for the IgG3 subclass than for the IgG1 subclass (Pleass and Woof 2001). The major B-cell epitopes recognized by these human IgG antibodies have been localized to conserved sequences in the GLURP₂₇₋₄₈₉ and MSP3₂₁₂₋₂₅₇ regions, respectively (Oeuvray, Bouharoun-Tayoun et al. 1994; Theisen, Soe et al. 2000; Theisen, Dodoo et al. 2001). Nucleotide-sequencing have demonstrated that these important epitopes are highly conserved among a number of P. falciparum laboratory lines and field isolates from Africa and Asia (Huber, Felger et al. 1997), (McColl and Anders 1997), (de Stricker, Vuust et al. 2000).

In view of the conservation of the epitopes, the inventors have investigated whether cytophilic antibodies against GLURP and MSP3 are involved in the development of immunity to clinical malaria in an Asian population of Myanmar, as they have been reported to be in Africa, i.e., in a different human and parasite genetic background. Since numerous reports have argued in favour of a direct role of antibodies against the C-terminus of MSP1, they also included this molecule in their investigations. Results, disclosed in Example 1 below, show that levels of cytophilic IgG3 antibodies against conserved regions of MSP3 and GLURP are significantly correlated with clinical protection against P. falciparum malaria. In contrast, levels of non-cytophilic IgG4 antibodies against GLURP increased with the number of malaria attacks. Most importantly, there was a complementary effect of the MSP3- and GLURP-specific IgG3 antibodies in malaria protection. In those individuals not responding to one of the antigens, a strong response to the other was consistently detected and associated with protection, suggesting that the induction of antibodies against both MSP3 and GLURP could be important for the development of protective immunity.

The inventors have produced a chimeric molecule comprising the GLURP₂₅₋₅₀₀ and MSP3₂₁₂₋₃₈₂ regions together as a recombinant hybrid-protein in Lactococcus lactis. This hybrid offers the possibility to investigate the vaccine potential of both antigens in single immunizations and can potentially increase the immunogenicity by combining a wider range of B and T helper cell epitopes (Example 2).

Different regions of these antigens have previously been produced in Escherichia coli fused to various affinity-tags (McColl, Silva et al. 1994; Theisen, Vuust et al. 1995; Theisen, Thomas et al. 2001). Whereas such additional sequences are advantageous for purification, they also pose a potential problem because host immune responses may be biased by foreign sequences. It is therefore desirable to explore expression systems, which aim to produce the recombinant protein without a vector-encoded affinity-tag. L. lactis was chosen as expression host because i) it is a well characterized industrial microorganism, generally recognized as safe (GRAS), best known for its use in the production of fermented dairy products, ii) it can be grown in a defined synthetic medium, iii) recombinant proteins are secreted into the culture supernatant, from where they can be easily purified, iv) it does not produce toxic substances. In this study, a novel gene expression system has been used, which is based on the pH and growth phase regulated promoter, P170, from L. lactis (Warmerdam, van de Winkel et al. 1991; Israelsen, Madsen et al. 1995; Madsen, Arnau et al. 1999). This gene expression system offers a simple fermentation procedure, which has been developed specifically for the P170 promoter. Results obtained by expressing the hybrid GLURP-MSP3 in this system tend to support both the value of the vector for vaccine development and the potential of the combination of the two parasite proteins. The results, disclosed in Example 2 below, show that mice immunized with the hybrid protein produced higher levels of both GLURP- and MSP3-specific antibodies than mice immunized with either GLURP, MSP3 or a mixture of both. The protective potential of the hybrid protein was also demonstrated by in vitro parasite-growth inhibition of mouse anti-GLURP-MSP3 IgG antibodies in a monocyte-dependent manner.

Throughout the following text, several terms are employed, the meaning of which should be understood according to the following definitions:

A “chimeric molecule” is a molecule comprising at least two moieties, wherein these moieties originate from two different biological molecules. The two different biological molecules can be two molecules of the same organism, even of the same strain. Particular examples of chimeric molecules exemplified below are fusion proteins comprising a moiety originating from the MSP3 antigen and a moiety originating from the GLURP protein of P. falciparum, but the two moieties can also be linked by any other means, provided they are linked covalently or by a non-covalent binding. For example, the two moieties, which can be peptidic moieties, can be both linked to a third element, for example a synthetic particle. A fusion protein is hence a particular example of chimeric molecule, but a chimeric molecule is not necessarily a fusion protein.

A “chimeric molecule” is also a composition consisting of an association of MSP3 protein and GLURP protein, wherein both proteins can be linked by covalent bounds, possibly by means of chemical spacers, but also possibly by non-covalent bonds

A “fusion protein” (also called “hybrid protein”, or sometimes merely “hybrid”) is a protein which comprises at least two moieties originating from different polypeptides or proteins. Of course, the two moieties must have a relevant size, i.e., they are not restricted to only a few amino acids. In the context of the present invention, each moiety is a polypeptide chain which advantageously comprises at least 20 amino acids and preferably, at least 50. Such a fusion protein is obtained by fusing the sequences coding for each of the peptidic moieties, in the same open reading frame, and having the resulting construct expressed. Of course, further elements, such as a third moiety coming from a third protein, or such as a linker, can be present in the fusion protein, in addition to the two moieties mentioned above.

A “conservative substitution” means, in an amino acid sequence, a substitution of one amino acid residue by another one which has similar properties having regard to hydrophobicity and/or steric hindrance, so that the tertiary structure of the polypeptide is not dramatically changed. For example, replacing a guanine by an alanine or vice-versa, is a conservative substitution. Valine, leucine and isoleucine are also amino acids that can be conservatively substituted by each other. Other groups of conservative substitution are, without being limitative, (D, E), (K, R), (N, Q), and (F, W, Y).

Further definitions are provided in the following text, when necessary.

A first object of the present invention is hence a chimeric molecule comprising a GLURP moiety consisting of a polypeptide fragment of at least 50 (preferably at least 150) amino acids from the GLURP₂₅₋₅₁₄ fragment of SEQ ID No:1, and a MSP3 moiety consisting of a polypeptide fragment of at least 50 (preferably at least 100) amino acids from the MSP3₂₁₂₋₃₈₀ fragment of SEQ ID No:2, or a variant thereof in which 1 to 15 amino acids in any or both of said moieties have been deleted, added or changed by conservative substitution, wherein said fusion protein raises antibodies against both the polypeptides of SEQ ID No: 1 and SEQ ID No: 2, in mice immunized with it.

An example of mouse model that can be immunized by a chimeric molecule to test the antibodies raised by said molecule, is the BALBc/CF1 strain used in Example 2. Any other mouse strain can be used for that purpose. The animal can be immunized with the chimeric molecule as described in example 2 below. An example of method to determine whether antibodies have been raised against both the polypeptides of SEQ ID No: 1 and SEQ ID No: 2 in said animal, is to collect serum from it and measure the anti-GLURP and anti-MSP3 antibody response by ELISA as described below. The result will be considered as positive (i.e., antibodies raised against the antigens), if the titers of both anti-GLURP and anti-MSP3 antibodies are superior to those observed in the sera collected from the same animals prior to their immunization plus twice the standard deviation, i.e.:
ODₛₐₘₚₗₑ>(OD_{normal serum}+2SD)

A preferred chimeric molecule according to the present invention is more immunogenic than a mixture of the polypeptides of SEQ ID No: 1 and SEQ ID No: 2. For example, such more immunogenic chimeric molecule raises in mice immunized with it higher levels of anti-MSP3 antibodies than either the MSP3₂₁₂₋₃₈₀ fragment of sequence SEQ ID No:2, or a mixture of both the GLURP₂₅₋₅₁₄ fragment of SEQ ID No:1 and the MSP3₂₁₂₋₃₈₀ fragment of sequence SEQ ID No:2.

This chimeric molecule can also or alternatively raise in mice immunized with it higher or equal levels of anti-GLURP antibodies than either the GLURP₂₅₋₅₁₄ fragment of SEQ ID No:1, or a mixture of both the GLURP₂₅₋₅₁₄ fragment of SEQ ID No:1 and the MSP3₂₁₂₋₃₈₀ fragment of sequence SEQ ID No:2.

Another property which can be sought in a chimeric molecule of the invention is that it raises in mammals immunized with it, IgG antibodies that can inhibit parasite growth in vitro in cooperation with human monocytes, as determined in an ADCI assay as described below. This can be tested, for example, in mice.

Generally, to determine whether a chimeric molecule has e of the properties mentioned above, the materials and methods described in Example 2 can be used.

In a particular embodiment of the invention, the chimeric molecule is a fusion protein. In the following text, reference will be made only to fusion proteins, for clarity reasons. However, when there is no technical limitation, the phrase “fusion protein” should be understood broadly as “chimeric molecule”.

In a particular fusion protein according to the invention, the GLURP moiety is N-terminal and the MSP3 moiety is C-terminal.

A fusion protein according to the invention can further comprise a linker having one to 50 amino acids between the GLURP and the MSP3 moieties. However, this linker will preferably be chosen so that it does not introduce a bias in the host immune response, i.e., so that an immune response against irrelevant foreign sequences is not significantly induced. This linker is preferably as short as possible. As mentioned above, chimeric molecules, in which the two moieties are linked by a non-peptidic linker, are also contemplated according to the present invention.

A particular fusion protein of the invention is the GLURP₂₇₋₅₀₀-MSP3₂₁₂₋₃₈₀ protein of SEQ ID No:3. As described in Example 2, mice immunized with this hybrid protein produced higher levels of both GLURP- and MSP3-specific antibodies than mice immunized with either GLURP, MSP3 or a mixture of both. The protective potential of the hybrid protein was also demonstrated by in vitro parasite-growth inhibition of mouse anti-GLURP-MSP3 IgG antibodies in a monocyte-dependent manner.

Another aspect of the invention concerns a recombinant nucleotide sequence encoding a fusion protein of the invention, as described above. For example, the nucleotide sequence of the invention can comprise the sequence of SEQ ID No: 4.

In a particular embodiment of the recombinant nucleotide sequence of the invention, the sequence encoding the fusion protein is under the control of a pH and/or growth phase regulated bacterial promoter which can be, for example, the P170 promoter from L. lactis.

The present invention also pertains to a recombinant, cloning and/or expression vector, comprising a nucleotide sequence as described above.

In a particular aspect of the invention, the chimeric molecule is bound to a support. The invention therefore also relates to the conjugates obtained by covalent coupling of the peptides according to the invention to physiologically acceptable and non-toxic (natural or synthetic) carrier molecules that enable, in particular, the immunogenicity to be increased, via complementary reactive groups carried, respectively, by the carrier molecule and the peptide. Natural proteins such as tetanus toxoid, ovalbumin, serum albumins, haemocyanins, tuberculin PPD (purified protein derivative), and the like may be mentioned as possible examples of macromolecular carrier molecules or supports which participate in the constitution of the conjugates according to the invention. Synthetic macromolecular supports can also be used, like for example, polylysines or poly(DL-alanine)-poly(-Lysine)s.

Hydrocarbon or lipid supports, for example saturated or unsaturated fatty acids, and preferably C₁₆ or C₁₈ acids of the oleyl or palmitoleyl type, can also be coupled to the antigenic peptides or polypeptides according to the invention. Conjugates consisting of a polypeptide originating from LSA-5 covalently linked via a lysine bridge to saturated or unsaturated lipid residues hence also form part of the invention, more especially when the lipid residue is a palmitoyl or a palmityl or an oleyl.

Lastly and without implied limitation, the molecules according to the invention may be coupled to traditional supports or adsorbed on such supports, in particular nitrocellulose, latex or polystyrene microspheres or beads, or incorporated in Tyl particles.

To synthesize the conjugates according to the invention, use may be made of methods with are known per se, such as the one described by Frantz and Robertson in Infect. And Immunity, 33, 193-198 (1981), or the one described in Applied and Environmental Microbiology (October 1981), vol. 42, No. 4, 611-614 by P. E. Kauffman, using the peptide and the appropriate carrier molecule.

The present invention also pertains to an “immunogenic composition comprising, as an immunogen, a fusion protein as described above. This immunogenic composition can be used, for example, to immunize animals for obtaining antibodies, or to immunize humans to protect them, at least partially, against malaria.

As described in Example 2, the fusion protein of the invention has advantageous immunogenic properties, and therefore can be used for the preparation of a vaccine composition against malaria. A vaccine against malaria, comprising as an immunogen a fusion protein according to the present invention, in association with a suitable pharmaceutical vehicle, is hence also part of the invention, as well as a method for vaccinating a subject against malaria, comprising administering to said subject a chimeric molecule as described above.

In Example 1, the inventors have shown that there is a complementary effect of the MSP-3 and GLURP-specific IgG3 antibodies in malaria protection. The induction of antibodies against both MSP3 and GLURP, or against fragments thereof, is hence a possible way towards the development of a protective immunity.

Other immunogenic compositions and vaccines according to the invention are hence characterized in that they comprise, as an immunogen, a mixture of GLURP and MSP3 antigens. By “mixture of GLURP and MSP3 antigens”, is meant that the compositions or vaccines comprise antigens from both GLURP and MSP3. These can be, for example, the native proteins, or antigenic fragments thereof, for example the MSP3b (a.a. 184 to 210) and/or MSP3d (a.a. 211 to 251) for MSP3, and the R0 GLURP fragment (a.a. 27 to 500).

In a particular embodiment of the immunogenic composition or vaccine: of the invention, said composition or vaccine further comprises: at least one antigen of Plasmodium falciparum selected amongst LSA-1 (Guerin-Marchand, Druilhe et al. 1987), LSA-3 (Daubersies, Thomas et al. 2000), LSA-5, SALSA (Bottius, BenMohamed et al. 1996), STARP (Fidock, Bottius et al. 1994), TRAP (Robson, Hall et al. 1988), PfEXP1 (Simmons, Woollett et al. 1987), CS (Dame, Williams et al. 1984), MSP1 (Miller, Roberts et al. 1993), MSP2 (Thomas, Carr et al. 1990), MSP4 (Marshall, Tieqiao et al. 1998, AF033037), MSP5 (Marshall, Tieqiao et al. 1998, AF033037), AMA-1 (Peterson, Marshall et al. 1989; Escalante, Grebert et al. 2001), and SERP (Knapp, Hundt et al. 1989).

The immunogenic composition or the vaccine according to the present invention can be formulated for intradermal or intramuscular injection. In this formulation, the composition or vaccine advantageously comprises from 1 to 100 μg of immunogen per injection dose, preferably from 2 to 50 μg.

Other elements, such as adjuvants, can be added to the immunogenic composition or vaccine of any the invention. Of course, usual adjuvants, such as Montanide and/or Alum, can be used therefor. Other possible adjuvants that can be used in the immunogenic composition or vaccine of the invention are described in EP 1 201 250 A1, such as SB62, SB26, and SBAS2 (AsO2). The vectors of the invention can be used for the preparation of a medicament for genetic immunisation against Plasmodium falciparum. Accordingly, the invention also pertains to a DNA vaccine comprising a nucleotide sequence as described above. For example Alternatively, the VR1020 vector (VICAL®), mentioned at least in (Kang, Calvo et al. 1998) and in (Valenzuela, Belkaid et al. 2001), can be used for obtaining constructs for direct DNA immunization.

A recombinant host cell, which is transformed (or recombined) by a vector according to the invention, is also part of the invention. This cell can be for example a bacterium, a yeast, an insect cell, or a mammalian cell.

Another aspect of the invention is a method for producing a recombinant protein, comprising a step of culture of a L. lactis bacterium containing, an expression vector comprising the coding sequence of said protein, under the control of a pH and/or growth phase regulated bacterial promoter, for example the P170 promoter from L. lactis, followed by a step of removal of the bacterial cells from the culture supernatant. This method has been successfully performed with the pKBR11 plasmid, as described in Example 2 below. This method can advantageously be performed with an htrA mutant strain, in order to avoid degradation products of the recombinant protein to be produced.

As shown in Example 3, passive transfer of anti-GLURP or anti-MSP3 antibodies in an immuno-compromised mouse model can clear the P. falciparum parasitemia. The present invention hence also pertains to the use of purified and/or recombinant anti-MSP3 and anti-GLURP antibodies or fragments thereof, for the preparation of a medicament against malaria. A medicament for passive immunotherapy of malaria, comprising purified and/or recombinant antibodies against MSP-3 and GLURP, or fragments thereof, is also part of the present invention. Preferred antibodies (or fragments) according to the invention are human or humanized antibodies. The purified antibodies can be obtained by any method known by the skilled artisan, from human or animal sera, by affinity-purification against the whole proteins or fragments thereof, etc. The recombinant antibodies or fragments of antibodies can be produced for example in Lemna, as well as in maize, tobacco, CHO cells, and the like. When produced in CHO cells, they can be obtained for example by using the method described in WO 03/016354. A mixture of two or more monoclonal antibodies, with at least one recognizing MSP3 and the other recognizing GLURP, can also be used for the preparation of a medicament for passive immunotherapy of malaria.

A method for lowering the parasitemia in a malarial patient; or for protecting against or treating Plasmodium falciparum in a subject presenting malarial symptoms, or likely to be infected by malaria, is also provided. Such a method consists in administering to said subject a medicament comprising purified anti-MSP3 and anti-GLURP antibodies.

Further aspects and details concerning the present invention will appear in the following experimental examples, which are illustrated by the figures.

### BRIEF DESCRIPTION OF THE DRAWINGS

FIG. 1: A. Pattern of IgG3 antibody responses against each of the antigens in the 30 protected individuals of OoDo (means and standard errors of the ratios of IgG3-specific responses). B. Pattern of IgG3 responses in 7 protected OoDo inhabitants with low IgG3 anti-MSP3 response (low IgG3 cut off values were defined as those under the lower 95% confidence interval limits of the mean, ie. anti-MSP3b IgG3 ratios <2.30). C Pattern of IgG3 responses in 15 protected OoDo inhabitants with low IgG3 anti-R0 response (low IgG3 cut off values were defined as those under the lower 95% confidence interval limits of the mean IgG3 ratios, ie. IgG3 ratios of anti-GLURP R0<1.38). D. Changes at 5 years interval in 7 protected individuals with high IgG3 MSP3 responses in 1998. E. Changes at 5 years interval in 6 protected individuals with high IgG3 anti-GLURP R0 responses in 1998.

FIG. 2: Schematic representation pf pPSM1013 and pAMJ328 and the expression constructs used in L. lactis. The position of vector encoded restriction sites mentioned in the text, promoter P170, Shine-Dalgarno sequence (SD), and 310mut2 signal peptide are indicated. The signal peptidase is predicted to cleave between amino acid no. 32 and 33, thus leaving Ala-Glu residues in the N-terminal end of the mature recombinant proteins. The nucleotide numbering of glurp and MSP3 was relative to A in the ATG codon of M59706 and L07944, respectively.

FIG. 3: Purification and sequence verification of the GLURP-MSP3 hybrid protein. (A) Coomassie blue-stained 12.5% polyacrylamide gel of purified GLURP-MSP3 fusion protein in peak 2 (lane 1), and peak 1 (lane 2), from the final Phenyl Sepharose High Performance column. The sizes (in kilodaltons) of the molecular mass markers are indicated. (B) List of tryptic peptides that match the GLURP-MSP3 hybrid protein. Amino acid residues are represented using the single letter codes and numbered according their positions in the deduced hybrid protein sequence. The molecular mass of peak 1 was 67688 Da (±70 Da) as determined by MALDI MS. (C) HPLC analysis on a C4 column of the GLURP-MSP3 hybrid protein and GLURP₂₅₋₅₁₄, respectively. (D) Coomassie blue-stained 12.5% polyacrylamide gel of purified GLURP-MSP3 fusion protein (lane 1), GLURP₂₅₋₅₁₄ (lane 2), and MSP3₂₁₂₋₃₈₀ (lane 3) produced in L. lactis MG1363. The equivalent of 250 μl Of culture supernatant was added to each well. The sizes (in kilodaltons) of the molecular mass markers are indicated.

FIG. 4: Patterns of IgG antibody responses to pairs of GLURP and MSP3 derived antigens in 71 plasma samples from adult Liberians clinically immune to malaria. The coefficient of correlation and P value are provided in each panel.

FIG. 5: Antibody responses in mice. Groups of 10 mice were immunized with the hybrid (gr7), a mixture of GLURP and MSP3 in one syringe (gr8), or with GLURP and MSP3 in separate syringes at different sites (gr9). (A) Day 35 plasma samples were tested for antibody reactivity on ELISA plates coated with GLURP₂₅₋₅₁₄ or MSP3₂₁₂₋₃. Box plots show medians, 25^{th}, and 75^{th} percentiles and whiskers show the range of the data. (B) Cumulative responses of mouse sera with 8 peptides representing GLURP B-cell epitopes (Theisen, Soe et al. 2000) and (C) isotype response of mice for which results are presented in panel A. Each vertical bar represents the mean absorbance (±SEM) in GLURP- and MSP3-specific ELISAs.

FIG. 6: The hybrid contains only GLURP and MSP3 derived B-cell epitopes. A pool of plasma from mice immunized with the hybrid was pre-incubated with GLURP, MSP3, a mixture of GLURP and MSP3 or the hybrid at the indicated concentrations before being added to ELISA coated with the hybrid. Prior incubation with a mixture of GLURP and MSP3 or the hybrid completely inhibited Ig antibody binding to the hybrid.

FIG. 7: Immunoblot analysis of P. falciparum NF54. A whole cell extract was separated on a 7.5% polyacrylamide gel and subjected to immunoblotting with plasma from mice immunized with GLURP₂₅₋₅₁₄ (lane 1), MSP3₂₁₂₋₃₈₀ (lane 2) and GLURP-MSP3 hybrid (lane 3). The sizes (in kilodaltons) of the molecular mass markers are indicated.

FIG. 8: Course of parasitemia in P.f.-HuRBC-BXN injected with human monocytes and treated with human IgG purified against the P falciparcum antigens EBA-175 and MSP3b.

FIG. 9: Course of parasitemia in P.f.-HuRBC-BXN injected with human monocytes and treated with human IgG purified against the P falciparum antigens MSP2 and MSP3-b.

FIG. 10: Course of parasitemia in P.f.-HuRBC-BXN injected with human monocytes and treated with human IgG purified against the P falciparum antigen MSP1.

FIG. 11: Course of parasitemia in P.f.-HuRBC-BXN injected with human monocytes and treated with human IgG purified against the P falciparum antigens GLURP and GLURP R0.

FIG. 12: Course of parasitemia in P.f.-HuRBC-BXN injected with human monocytes and treated with human IgG purified against the P falciparum antigen MSP3-d.

FIG. 13: Course of parasitemia in P.f.-HuRBC-BXN injected with human monocytes and treated with human IgG purified against the P falciparum antigen AMA-1.

### EXAMPLES

### Example 1

### Complementarity Between Responses to MSP3 and GLURP Shown in a Longitudinal Clinical and Parasitological Follow-Up Study

1.A. Materials and Methods

1.A.1. Study Area, Population and Clinical Surveillance

OoDo village is a re-settled forested region of Myanmar with a tropical climate characterized by hot dry, monsoon and cool dry seasons. In this area, malaria was found to be stable and hyper-endemic with seasonal variation, the majority of infections were due to Plasmodium falciparum (98%) and Plasmodium vivax was responsible for the remaining 2%. A malaria attack was defined according to 4 concomitant criteria: i)—corrected axillary temperature ≧38.0° C., ii)—absence of other clinical diseases, iii)—presence of asexual P. falciparum forms in thick-films, and iv)—clinical and parasitological improvement after chloroquine treatment. Two febrile attacks were regarded as two different malaria episodes if they were separated by ≧72 h. The results of the first 33 months of follow-up have recently been published (Soe, Khin Saw et al. 2001). The same study population was followed-up for one additional year, up to 31^{st} December 1998, using the same protocol. Venous blood samples were drawn during September 1998, and malarial attack rates recorded from Jan. 1^{st} to Dec. 31^{st} 1998 were used to analyze the relationship with clinical protection.

1.A.2. Blood Sampling and Parasitological Study

Surveillance of malarial infection was carried out by systematic monthly examination of thick and thin blood films from finger-prick. A slide was regarded as negative if no parasite was visualized in 200 oil-fields in Giemsa stained thick film. For febrile cases two finger-prick films before and after chloroquine treatment were examined. Venous samples were collected in vacutainers, sera aliquoted aseptically, and stored at −20° C. until tested. Samples taken from a representative subgroup of 116 villagers from whom more than 60% of the monthly blood films were available for parasitological data were selected from the larger cohort of 292 residents.

1.A.3. Antigens

The three recombinant GLURP antigens were derived from the N-terminal non-repeat region R0 (GLURP₂₇₋₅₀₀), the central repeat region R1 (GLURP₄₈₉₋₇₀₅), and the C-terminal repeat region R2′ (GLURP₇₀₅₋₁₁₇₈) of P. falciparum F32 (Oeuvray, Theisen et al. 2000). The C-terminal 19-kDa fragment of MSP1, MSP1₁₉, from the Wellcome strain (MSP1-W-19) was produced as a recombinant GST-fusion protein in Eschenchia coli and was a kind gift from Dr. A. Holder, UK. The GST-tag was removed by enzymatic cleavage and subsequent affinity chromatography before use. The MSP3b synthetic peptide (184-AKEASSYDYILGWEFGGGVPEHKKEEN-210, SEQ ID No:5) contained the MSP3b B-cell epitope which reacts with ADCI-effective human antibodies (Oeuvray, Bouharoun-Tayoun et al. 1994).

1.A.4. Antibody Assays

The levels of antibodies to the three P. falciparum-derived antigens were measured by enzyme-linked immunosorbent assay (ELISA) as previously described (Oeuvray, Theisen et al. 2000). Briefly, microtiter plates (Maxisorb, Nunc, Denmark) were coated overnight at 4° C. with recombinant proteins or synthetic peptide at the following concentrations: 0.5 μg/ml (R0 and R2), 1 μg/ml (R1 and MSP1) and 5 μg/ml (MSP3b). For GLURP antigens 0.05 M Na₂CO₃, pH 9.6 and for MSP1 and MSP3 phosphate buffered saline (PBS) pH 7.4 were used as coating buffers. The next day the plates were washed with PBS plus 0.05% Tween 20 (PBST) and blocked with 2.5% non-fat milk in PBS for 2 h. Sera diluted in PBST containing 1.25% (w/v) non-fat milk, were added to duplicate wells and incubated for 1 h at room temperature. Various dilutions of sera were made for each antigen: 1:200 for GLURP, 1:100 for MSP1 and 1:20 for MSP3. These dilutions were selected after preliminary pilot studies, which revealed more than a 10-fold difference between control and test samples. Bound antibody was detected by peroxidase-conjugated goat anti-human immunoglobulin (Caltag Laboratories), diluted 1:3000. Color was revealed by O-phenylenediamine (Sigma, St. Louis, Mo.) and H₂O₂ in citrate buffer pH 5 for 30 min. The optical density (OD) at 492 nm was determined in a plate reader (Titertek Multiskan MCC 1340). The plates were washed extensively with PBST between each incubation step. All ELISA tests included 6 control sera, randomly selected among 100 French blood donors never exposed to malaria.

For subclasses determination of IgG1-4, monoclonal mouse anti-human subclasses (clones NL16=IgG1 (Boehringer®), HP6002=IgG2 (Sigma®), Zg4=IgG3, and RJ4=IgG4 (both from Immunotech®)) were used. They were diluted 1:2,000, 1:10,000, 1:10,000, and 1:1,000, respectively in 1.25% (w/v) non-fat milk in PBST and incubated for 1 h at room temperature. Goat anti-mouse IgG conjugated to peroxidase (Caltag Laboratories®D), diluted 1:3000 in 1.25% (w/v) non-fat milk in PBST was added and incubated for 1 h. Bound labeled antibody was revealed as described above. The dilutions of each isotype-specific monoclonal antibody (MAb) had been determined previously as those discriminating between human Ig sub-classes, i.e. yielding no cross-reactions between subclasses (Oeuvray, Theisen et al. 2000). The results for total IgG as well as subclass antibody levels were expressed as ratios of antibody response which were calculated by dividing the mean OD of test with the mean plus 3 SD of the 6-normal controls run simultaneously. A sample with a ratio of ≧1 was considered positive.

1.A.5. Statistical Analysis

The Mann-Whitney U-test and Spearman's rank-order correlation coefficient were used for the calculations of P-values. The association between the risk of malaria attack during 1998 and the levels of antibodies (expressed in ratios) were tested with JMP® software, using either a Poisson regression model where the effect of confounding factors such as age, gender, time spent in the village and transmission were controlled or a logistic regression analysis (with or without occurrence of malaria attack).

1.B. Results

1.B.1. P. falciparum Infections in the Study Cohort

All 116 subjects in the study cohort were from OoDo village situated in Myanmar, South-East Asia, where malaria is hyper-endemic (Soe, Khin Saw et al. 2001). The prevalence of P. falciparum parasitemia fluctuated around 40% from January to July and dropped to around 20% from August to December in 1998. The incidence of clinical malaria, which was calculated as the average number of attacks per month in the study cohort and expressed in percentage varied considerably over the year, peaking in June. The infective inoculation rate has not been determined for OoDo, however, Tun-Lin, W et al (Tun-Lin, Thu et al. 1995) found 13.7 infective bites per person per year in a village, which is located 15 km East of OoDo village. The finding agrees well with an estimated number of 11 infective bites per person per year as calculated by the method of (Beier, Killeen et al. 1999). Most infections (98%) were due to P. falciparum (Soe, Khin Saw et al. 2001). During the 12-month period of continuous clinical surveillance, 86 (74%) of the 116 villagers had at least one malaria attack as defined in the Material and Method section and these individuals were considered to be susceptible to malaria. During the same 12-month period, 30 (26%) of the villagers had no episode of clinical malaria, and these individuals were regarded as clinically protected.

1.B.2. Antibody Recognition of P. falciparum-Derived MSP3, GLURP, and MSP1 Antigens

Levels of IgG, and IgG subclasses against the MSP3b₁₈₄₋₂₁₀ peptide (MSP3b) and the four recombinant proteins representing the GLURP₂₇₋₅₀₀ (R0), GLURP₄₈₉₋₇₀₅ (R1), GLURP₇₀₅₋₁₁₇₉ (R2), and MSP1-19-kDa C-terminal regions were determined in the 116 sera collected during September 1998. R2 was the most frequently recognized antigen by IgG antibodies (67.2%) followed by R1, MSP3b and MSP1 (all at 62%), and R0 (58.6%). The highest OD values were obtained against R0 and R2, whereas MSP3b yielded lower OD values. Levels of IgG against all three GLURP regions and MSP1 were significantly associated with age (Spearman's rank-order correlation coefficient, R=0.51, 0.26, 0.41, and 0.43 for R0, R1, R2, and MSP1, respectively, P<0.05) while the IgG response against MSP3 was independent of age (R=0.16, P=0.17). As for the subclass responses, IgG1 and IgG4 against MSP1, IgG2 against MSP3, IgG1 and IgG3 against R0, IgG2 and IgG3 against R1 and IgG4 against R2, were found significantly associated with age (Table 1). Neither level nor prevalence of positive antibody response varied with gender for any of the antigens tested.

TABLE 1
Relationship between age and the level of subclass antibody responses to
each of the antigens studied. P and R-values were calculated by the
Sperman's Rank correlation Coefficient.

| | IgG Subclasses | P | R |
|---|---|---|---|
| Antigens | | | |
| MSP1 | IgG1 | 0.0002 | 0.344 |
| | IgG4 | 0.0009 | 0.309 |
| MSP3 | IgG2 | 0.0090 | 0.242 |
| GLURP antigens | | | |
| R0 | IgG1 | 0.0220 | 0.213 |
| | IgG3 | 0.0040 | 0.268 |
| R1 | IgG2 | 0.0040 | 0.268 |
| | IgG3 | 0.0008 | 0.313 |
| R2 | IgG4 | 0.0140 | 0.231 |

1.B.3. Antibody Responses and Clinical Protection

A striking difference between IgG subclass responses and protection was observed for the three different antigens (Table 2). For example, the IgG response against the C-terminal 19-kDa fragment of MSP1 was almost exclusively of the IgG1 subclass with a median value 8.6 times higher in the protected than in the susceptible group whereas, IgG3 antibodies predominated against the MSP3b epitope in protected individuals with a median value 6.5 times higher than that found in susceptible individuals. Although less pronounced, a similar dissimilarity in the cytophilic IgG subclasses response was also observed for different regions of GLURP, IgG1 antibodies predominating against the non-repeat R0-region and IgG3 antibodies prevailing against the R2 repeat-region.

TABLE 2
Median levels (and interquartile range) of IgG subclass antibodies to
MSP1, MSP3 and GLURP antigens found in villagers from OoDo
considered as either protected from, or susceptible to, P. falciparum
malaria attacks over 1 year of active and continuous follow-up.

| | | | | | Fold |
|---|---|---|---|---|---|
| | IgG | Protected | Susceptible | P | dif- |
| Antigen | Subclass | group (n = 30) | group (n = 86) | values | ference |
| MSP1 | IgG1 | 9.5 (0.8-25.03) | 1.1 (0.5-8.22) | .003# | 8.6 |
| | IgG2 | 0.9 (0.8-1.26) | 0.9 (0.8-1.09) | >.05 | |
| | IgG3 | 0.9 (0.1-2.63) | 0.4 (0.0-0.81) | >.05 | |
| | IgG4 | 1.2 (0.8-3.01) | 1.0 (0.7-1.47) | .01 | |
| MSP3 | IgG1 | 1.4 (0.8-2.4) | 0.7 (0.4-7.0) | <.001* | 2.0 |
| | IgG2 | 1.1 (0.9-1.57) | 0.8 (0.7-1.0) | >.05 | |
| | IgG3 | 6.5 (2.5-14.03) | 1.0 (0.6-1.49) | <.001* | 6.5 |
| | IgG4 | 1.3 (1.0-1.82) | 1.0 (0.9-1.35) | <.001* | 1.3 |
| R0 | IgG1 | 3.9 (2.4-7.62) | 1.8 (1.0-3.15) | <.001* | 2.2 |
| | IgG2 | 0.9 (0.4-2.5) | 0.8 (0.4-1.33) | >.05 | |
| | IgG3 | 1.3 (0.6-3.76) | 0.9 (0.3-1.44) | .019 | |
| | IgG4 | 0.2 (0.2-2.05) | 0.6 (0.2-1.07) | >.05 | |
| R1 | IgG1 | 0.3 (0.1-0.9) | 0.2 (0.1-0.4) | >.05 | |
| | IgG2 | 0.9 (0.4-1.64) | 0.6 (0.4-0.91) | >.05 | |
| | IgG3 | 1.2 (0.4-2.64) | 0.5 (0.2-0.91) | .039 | |
| | IgG4 | 0.2 (0.2-0.52) | 0.7 (0.2-0.94) | .021 | |
| R2 | IgG1 | 2.0 (0.9-5.4) | 1.1 (0.3-3.11) | .01 | |
| | IgG2 | 2.0 (1.0-4.02) | 0.9 (0.2-1.73) | <.001* | 2.2 |
| | IgG3 | 6.4 (1.7-12.01) | 0.9 (0.3-2.83) | <.001* | 7.1 |
| | IgG4 | 1.0 (0.6-1.2) | 0.6 (0.4-0.85) | .003 | |

Given the number of statistical tests carried out, the Bonferroni's correction factor was applied to determine the level of significance and only P values <.0025 were considered significant (*).
P values were determined from the non-parametric Mann-Whitney U-test. Fold difference refers to the ratio of median values from the two groups.
#Values marginally different.

Since the antibody titers against GLURP and MSP1 increased as a function of age, the correlation of clinical status of the villagers with various antibodies was reexamined in a logistic-regression model considering age and all the antibody responses (log transformed) as explanatory variables. When testing these parameters in the model and in particular when age was controlled for, among all antibody responses, the strongest predictors of malaria protection identified were increased levels of IgG3 against MSP3b (F ratio=67.5; P<0.0001) and against GLURP-R0 (F ratio=23.1; P<0.0001). Other antibodies were not significantly associated with protection. In contrast, the analysis indicated that levels of IgG4 against R0 (F ratio=4.4; P=0.038) and R1 (F ratio=3.9; P=0.051) increased with the number of malaria attacks, i.e. they were to some extent predictive of susceptibility to malaria.

1.B.4. Antigen Specificity of IgG3 Responses in Protected Villagers

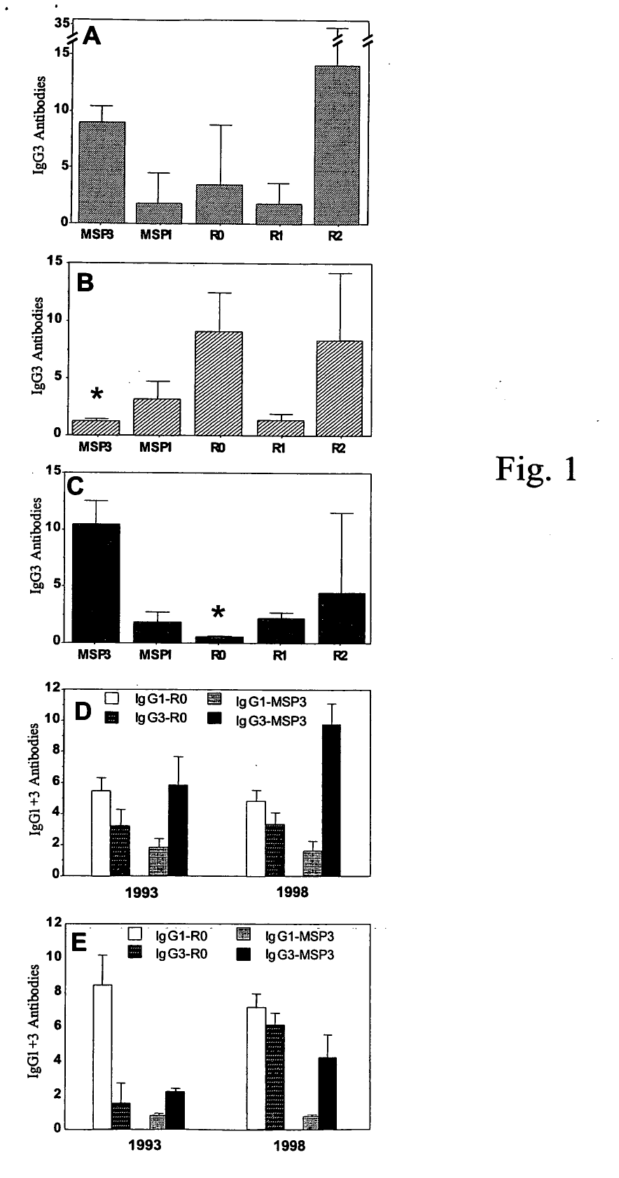

FIG. 1A shows the general pattern of IgG3 antibody responses found against the different blood stage antigens in OoDo. The range of values was large for most antigen-specific antibody responses and this suggested that different subgroups of “responders” might exist. Sera of villagers who were protected from clinical malaria did not all show high IgG3 values against both MSP3b and GLURP-R0. Some individuals displayed an unexpectedly low IgG3 reactivity against either one of these 2 antigens. In an attempt to understand how these villagers were protected against malaria attacks, two sub-groups were identified, characterized by: a)—low IgG3 responses against MSP3 (7 out of 30 cases) or b)—low IgG3 responses against R0 (15 out of 30 cases). The levels of IgG3 antibodies against the other three antigens were estimated (FIG. 1B). The 7 protected individuals (mean age±1std error=33.9±7.0 years) with a low IgG3 response to MSP3b (IgG3 ratio=1.26±0.22) were found to have a strong IgG3 response to R0 (IgG3 ratio=9.09±3.41) and to R2 (IgG3 ratio=8.36±5.86). In the 2^{nd} subgroup of 15 other individuals (24.7±4.3 years of age) also protected despite a low IgG3 response to GLURP-R0 (IgG3 ratio=0.55±0.08), the reverse situation was found (FIG. 1C): a high IgG3 antibody response against MSP3b was observed (IgG3 ratio=10.45±2.07) and to a lesser extent against GLURP-R2 (IgG3 ratio=4.43±0.80). The titers for those responding to only one antigen tended to be higher than those responding to both antigens (Table 2). The number of years e not significantly differ between the groups of low-responders to MSP3 (20.43±4.70 years of residence) and R0 (18.7±10.6 years of residence).

Sera from 13 of the 30 individuals considered as protected in 1998 had also been sampled in 1993, and therefore were used to compare at 5 years interval the relative levels of anti-MSP3 and anti-R0 IgG1 and IgG3 antibodies. As shown in FIGS. 1D and 1E, there was no major change detectable in the levels of specific IgG1 against the two antigens. In contrast, levels of IgG3 antibodies to both MSP3 and GLURP increased from 1993 to 1998. In the subgroup of 7 individuals with elevated IgG3 against MSP3 in 1998 (FIG. 1D), the difference corresponded to a 1.67 times increase (P=0.11). In the subgroup of 6 subjects with elevated IgG3 against R0 in 1998 (FIG. 1E), the difference corresponded to a more important, ie. 3.93 times increase (P=0.05). The 6 individuals with high IgG3 responses against MSP3 in 1998 also had high titers 5 years earlier, suggesting that they were already protected via a sustained anti-MSP3 IgG3 response in 1993, when they were 18.2±9.8 years of age. In contrast, for GLURP the 7 individuals with a strong anti-R0 IgG3 response detectable in 1998 had substantially lower anti-R0 IgG3 responses 5 years earlier (P=0.0157), when they were 23.7±6.9 years of age. Thus, there was a drastic change in those 7 individuals protected via IgG3 to R0 in 1998 and their protection 5 years earlier was possibly related to IgG3 against MSP3.

1.C. Discussion

The present study is the first one to show an association between antigen-specific antibody responses and protection from clinical malaria in SE Asia. The prevalence of positive antibody responses against GLURP and MSP3 was high in OoDo, ranging from 58.6% (R0) to 67.2% (R2). This observation is in-keeping with the finding that B-cell epitopes within GLURP and MSP3 are highly conserved among P. falciparum laboratory lines and field isolates from Africa and Asia (Huber, Felger et al. 1997), (McColl and Anders 1997; de Stricker, Vuust et al. 2000). The prevalence of antibodies to MSP1-W-19 was also high, being almost twice the values found in the Gambia and Sierra Leone (Egan, Morris et al. 1996) and in Ghana (Dodoo, Theander et al. 1999) suggesting that strains related to the Wellcome strain might be prevalent in OoDo.

The highest ELISA-titers were found against the recombinant GLURP R0- and R2-regions and MSP1. The differences in GLURP-R0, -R1, -R2 and MSP1 ELISA-titers very likely reflected differences in serum antibody reactivity. In contrast, the MSP3-ELISA gave comparatively lower signals, however this discrepancy might be at least in part, related to the use of a short synthetic peptide defining a single or limited number of epitopes, as compared to recombinant proteins in the case of GLURP and MSP1 which are known to define several epitopes (Theisen, Soe et al. 2000).

Levels of IgG against all GLURP regions and MSP1 were significantly associated with age (P<0.05) while in contrast to this situation, the IgG response against MSP3 was found independent of age. Regarding IgG subclass responses, several of them were also found significantly associated with age and these variations could reflect the duration of exposure to the malaria parasites as well as the gradual maturation of the immune system over time.

There was a statistically significant increase in the levels of IgG3 against R0 and MSP3 among the protected individuals living in OoDo as compared to the non-protected ones. These results are in agreement with those of Dodoo et al. (Dodoo, Theisen et al. 2000), who found that cytophilic antibody responses against R0 and R2 were strong predictors of protection in Ghanaian children, and those of Oeuvray et al., who found a consistent correlation between protection and elevated IgG3 against both GLURP-R0 and R2 in Dielmo, West Africa (Oeuvray, Theisen et al. 2000). Similarly MSP3-specific IgG3 responses have previously been associated with protection against clinical malaria in Dielmo. Altogether, these results suggest that the same subclass of IgG response to the same critical epitopes are involved in the gradual development of protection against P. falciparum malaria in African as well as in Asian populations living in malaria endemic areas. In addition, the present study found a significant negative correlation between the levels of non-cytophilic antibodies against R0 and R1 and clinical protection. Therefore, on the one hand, there is a positive association between cytophilic IgG subclass responses and protection and on the other hand, a negative association between non-cytophilic subclass responses with the same epitope specificity and protection. This epidemiological result is in agreement with the in vitro observation that non-cytophilic antibodies can inhibit the bridging of merozoites and human monocytes by cytophilic antibodies against the same antigenic target and thereby reduce the ability of the latter to control parasite multiplication by the ADCI mechanism (Bouharoun-Tayoun and Druilhe 1992).

Whereas most of the protected residents of OoDo had high IgG3 responses against both MSP3 and GLURP, a number of individuals with low or almost no IgG3 responses against either one of these antigens also appeared to be protected. The inventors found that all the protected individuals with low GLURP-R0 specific IgG3 response had significantly elevated levels of specific anti-MSP3 IgG3 antibodies, and vice versa. This observation suggests that antibodies against GLURP and MSP3 may act in a complementary manner to control parasite multiplication in immune individuals. This is relevant in consideration of the role of these antibodies in ADCI mechanism. Indeed, only the simultaneous assessment of several antigens disclosed this complementary effect. This finding is in favor of testing simultaneously several antigens for complementary as for possible antagonistic effects that could have consequences on the design of combined vaccines.

In conclusion, the present study shows that (1)—the critical epitopes in the MSP3 and GLURP antigens which are most conserved, are targets of protective antibodies in geographically distant endemic areas of the world. (2)—IgG3 antibodies to MSP3 and GLURP-R0 are the strongest predictors of protection from clinical malaria in an African and also an Asian setting. (3)—To reach a state of premunition in Asia as well as in Africa, it is needed to produce a cytophilic subclass of antibody against critical antigens (namely, MSP3b and GLURP which both induce antibodies active in ADCI).

(4)—There appears to be a complementation effect between these two antigens. IgG3 responses might have similar effects against the risk of malarial attacks, provided they are present against one antigen when responses to the other are low or almost absent. (5)—The responses to different B cell epitopes on a given antigen appear to evolve independantly and the level of recognition can change over time.

The complementarity of responses observed to the two main targets of ADCI identified to date provide the first rational basis for combining these two antigens in a hybrid vaccine formulation. Moreover, immunogenicity studies performed in pre-clinical animal models with the hybrid vaccine lend further support to this antigen combination by showing improved immunogenicity with well-balanced, equilibrated responses to each molecule (example 2).

### Example 2

### A Plasmodium falciparum GLURP-MSP3 Chimeric Protein: Expression in Lactococcus lactis, Immunogenicity and Induction of Biologically active antibodies

2.A. Materials and Methods

2.A.1. Bacterial Strains, Plasmids and Growth Conditions

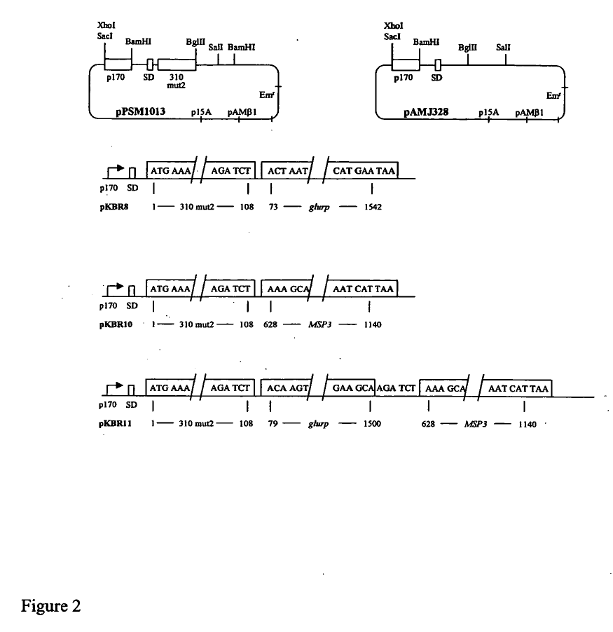

E. coli DH10B (K-12, F⁻ mcrA Δ(mrr-hsdRMS-mcrBC) φ80dlacZ Δm15ΔlacX74 deoRrecA1 endA1 araD139 Δ(ara, leu)7697 galU galK λ rpsL nupG) (Life Technologies®) containing the indicated plasmids was grown in Luria broth (LB) supplemented with erythromycin (200 μg/ml). L. lactis MG1363 (Gasson 1983) containing the indicated plasmids was grown in either M17 broth (Difco Ltd.®) with 0.5% (wt/vol) glucose or an enhanced synthetic amino acid (SA) medium named 3×SA IV medium supplemented with 1 μg/ml of erythromycin. Solidified LB or M17 media was supplemented with 200 or 1 μg/ml of erythromycin, respectively. The vector, pPSM1013 (FIG. 2), is a high-copy number expression plasmid based on the pAMβ1 replicon (Simon and Chopin 1988) containing unique restriction sites allowing the construction of in-frame fusions with an optimized secretion signal-peptide sequence, SP310mut2 (Ravn, P., Arnau, J., Madsen, S. M., Vrang, A., and Israelsen, H. unpublished). The mRNA for the peptide is translated from a plasmid-encoded translational start site and transcribed from the pH and growth phase inducible L. lactis promoter, P170 (Israelsen, Madsen et al. 1995; Madsen, Arnau et al. 1999). There is essentially no transcription from the P170 promoter at pH values of 7 or more. However, the transcription is induced in the transition to stationary phase at pH values below 6.5. Plasmid pAMJ328 is derived from pPSM1013 by deleting all lacZ regulatory sequences to avoid transcription from the lac promoter and by creating a new cloning region devoid of the signal peptide.

2.A.2. Construction of Plasmids Expressing GLURP and MSP3 in L. lactis

All plasmids were constructed in E. coli DH10B and transformed into L. lactis MG1363 by electroporation as described (Holo and Nes 1995). All plasmid constructions were verified by DNA sequencing.

pMST73. The non-repeat region of FVO glurp was amplified with the primers 5′-CCC AGA TCT ACA AGT GAG AAT AGA AAT AAA C (nucleotides 79 to 100, counting from A in the ATG start codon of the sequence of GenBank accession number M59706) and 5′-CCC AGA TCT TGC TTC ATG CTC GCT TTT TT CCG AT (nucleotides 1475 to 1500 of the sequence of GenBank accession number M59706); digested with BglII, and the resulting DNA fragment was cloned into BglII digested pPSM1013.

pKBR5. pMST73 plasmid was digested with BamHI and SalI, and the resulting DNA fragment containing the glurp insert was cloned into BamHI-SalI digested pAMJ328.

pKBR7. The non-repeat region of F32 glurp was amplified with the primers 5′-AAG TAG ATC TAC TAA TAC AAG TGA GAA TAG AAA TAA AC (nucleotides 73 to 100), and 5′-GTT CAG ATC TTT ATT CAT GAT GGC CTT CTA GC (nucleotides 1519 to 1542); the resulting DNA fragment digested with BglII and cloned into BglII digested pPSM1013.

pKBR8. Plasmid pKBR7 was digested with BamHI and SalI, and the glurp insert was cloned into BamHI-SalI digested pAMJ328.

pKBR9. The C-terminal region of F32 MSP3 was amplified with the primers 5′-CCC AGA TCT AAA GCA AAA GAA GCT TCT AGT TAT (nucleotides 628 to 651) and 5′-ATT AGA TCT CAT TTA ATG ATT T AAA ATA UT GGA TA, (nucleotides 1118 to 1140, counting from A in the ATG start codon of L07944); the resulting DNA fragment was digested with BglII and cloned into BglII digested pPSM1013. This MSP3 region is identical to that of the FC27 allele (Genbank accession number L07944) except for the following residues at variable positions in MSP3: 735 (T→C) and 948 (A→G).

pKBR10. Plasmid pKBR9 was digested with BamHI and SalI, and the MSP3 insert was cloned into BamHI-SalI digested pAMJ328.

pKBR11. The BglII-fragment of pKBR9 was cloned into pKBR5 digested partially with BglII yielding an in frame fusion between glurp₇₉₋₁₅₀₀ and MSP3₆₂₈₋₁₁₄₀. This hybrid molecule corresponds to the F32 allele except for the following residues at variable positions in GLURP: Leu-50, Asn-53, Glu-65, Asp-129, Glu-224, Pro-500.

2.A.3. Fermentation.

Fermentation of L. lactis MG1363, containing plasmid pKBR8 (GLURP), pKBR10 (MSP3) or pKBR11 (GLURP-MSP3 hybrid), was carried out in 1 L of 3×SA IV-media supplemented with erythromycin (1 μg/ml), yeast-extract (0.5%) and glucose (1.5%) in 2 L fermentors at 30° C. The starting pH of the culture medium was adjusted to 7.4. Since L. lactis MG1363 produces lactic acid during the growth, pH is declining as cell density increases. After approximately 3 hours of growth, pH was reduced to 6 and this level was maintained by a pH-controlled intake of 2 M KOH for another 8 hours until the cell density was approximately OD₆₀₀=8. A 50% glucose solution was added in parallel with the base since this tends to increase the bacterial yield. Bacterial cells were removed from the culture-supernatant (containing exported protein) by ultrafiltration with a Pellicon 2 Durapore filter (PVDF, 0.22 μm, 0.1 m2) (Millipore®). Culture-supernatants were either used immediately or stored at −20° C.

2.A.4. Purification of Recombinant Proteins

Cell-free culture-supernatants were concentrated on a Millipore Labscale™ TFF System installed with a Pellicon XL Biomax 8 filter (Polypropylene-membrane, 50000 Da, 50 cm²) and concentrates were buffer exchanged to 20 mM Bis-Tris (pH 6.4) on a Sephadex G-25 column (C26/40, 170 ml). Recombinant proteins were first purified on a 5 ml HiTrap Q Sepharose High Performance (Phamacia Biotech®) column by applying a gradient of 0 to 1 M NaCl in column buffer at a flow-rate of 1 ml/min. Fractions (2 ml) containing the desired recombinant protein were pooled and dialyzed against 20 mM Bis-Tris (pH 6.4) and applied to a 5 ml HiTrap SP Sepharose High Performance (Phamacia Biotech®) column. The recombinant protein was eluted by a gradient of 0 to 1 M NaCl in column buffer. GLURP and MSP3 were eluted in single peaks whereas the hybrid was eluted in two peaks. Fractions (2 ml) containing the first peak of the GLURP-MSP3 hybrid protein were pooled and adjusted to 1 M (NH₄)₂SO₄ and further purified on a 5 ml Phenyl Sepharose High Performance (Phamacia Biotech®) by applying a gradient of 1 to 0 M (NH₄)₂SO₄ in 20 mM Bis-Tris (pH 6.4) at a flow-rate of 1 ml/min. Analysis of all fractions was performed by SDS-PAGE. Protein concentrations were measured by the BCA™ protein assay (Pierce, Rockford, Ill., USA).

2.A.5. Immunization and Purification of Mouse IgG

Thirty BALBc/CF1 mice (Klausen, Magnusson et al. 1994) female mice (7 to 10 weeks of age) were randomly assigned to three groups. Two groups were immunized with 20 μg of GLURP₂₇₋₅₀₀-MSP3₂₁₂₋₃₈₀ hybrid (gr7), or with a mixture of 15 μg GLURP₂₅₋₅₁₂ and 5 μg MSP3₂₁₂₋₃₈₀ (gr8) by subcutaneous injections at the base of the tail, respectively; and the third group (gr9) received 15 μg GLURP₂₅₋₅₁₂ injected at the base of the tail and 5 μg MSP3₂₁₂₋₃₈₀ injected in the shoulder. All immunogens were emulsified in Montanide ISA720 and each mouse received three injections at 2-week intervals and was bleed on days 0, 14, 28 and 35. Total IgG was purified by (NH₄)₂SO₄ precipitation and subsequent purification on DEAE-columns from pooled serum samples taken on day 35 from animals in the groups gr7, 8, and 9 and from pooled day 0 samples.

2.A.6. ELISA and Serum Samples

Enzyme-linked immunosorbent assays (ELISAs) were performed as previously described in detail (Theisen, Vuust et al. 1995). The coating concentrations of GLURP₂₅₋₅₁₂, MSP3₂₁₂₋₃₈₀, and GLURP₂₇₋₅₀₀-MSP3₂₁₂₋₃₈₀ were 0.5, 1.0 and 0.5 μg/ml, respectively. Serial dilutions of plasma from Liberian adults clinically immune to malaria, Danish donors never exposed to malaria (Theisen, Soe et al. 2000), and mice were tested on ELISA plates coated with either antigen and the absorbance values were plotted against the plasma dilutions. In order to compare anti-hybrid antibody responses with the respective anti-GLURP and anti-MSP3 antibody responses in different plasma samples the antibody titer was defined as the plasma dilution, which gives an absorbance value of A492=1000 in the parallel portion of the curves.

2.A.7. Competition ELISA Assays

Recombinant GLURP₂₅₋₅₁₈ and MSP3₂₁₂₋₃₈₀ and a mixture of these two antigens were added at various concentrations (3.2×10⁻⁵ μg/ml to 100 μg/ml) to a pool of plasma from mice immunized with the GLURP-MSP3 hybrid diluted in 1.25% (w/v) milk powder in PBS. The plasma dilution used was adjusted to give an absorbance (A492) of approximately 2500. The antigen-antibody mixtures were incubated overnight at 4° C. and subsequently the reactivity to GLURP-MSP3 hybrid coated ELISA plates was determined.

2.A.8. Indirect Immunofluorescent Antibody (IFA) Test

IFA was performed as reported earlier (Bouharoun-Tayoun, Attanath et al. 1990). Briefly, a thin film of RBCs containing predominantly schizonts stages of P. falciparum NF54 were incubated with serial dilutions of purified mouse IgG in phosphate buffered saline (PBS pH 7.4) for 30 min at 37° C. in a humid chamber. After washing with PBS, mouse antibodies were revealed with Alexa Fluor conjugated goat anti-mouse IgG (Molecular probe, USA) diluted 1:300 in PBS. After washing the slide was examined under UV light. The endpoint titre was the highest dilution of the antibodies, which produce visible specific immunofluorescence.

2.A.9. Direct Inhibition (DI) and Antibody Dependent Cellular Inhibition (ADCI) Assays

Peripheral blood mononuclear cells were isolated from healthy European blood donors, without previous exposure, to malaria, by density gradient separation on J PREP (TechGen®) and subsequently added to 96 wells flat-bottom culture plates (TPP®, Switzerland). Each well was washed three times with RPMI, thereby separating non-adherent mononuclear cells from the attached monocytes (2×10⁵ monocytes per well). Mature schizonts from fast-growing synchronized in vitro culture of P. falciparum NF54 were separated by floatation over 50% Plasmagel, diluted with fresh erythrocytes to a final haematocrit of 2% and a parasitemia of 0.5%, and then added to each well. Purified mouse IgG was dialyzed for 48 hours against RPMI and added to the wells at three different concentrations. The final volume in each well was adjusted to 100 μl with RPMI supplemented with 0.5% Albumax. Similar cultures without Monocytes were performed in parallel to assess the DI. In all experiments the following controls were run simultaneously on each Plate: i) normal mouse IgG (NIG) from non-immunized mice to assess the nonspecific inhibitory components introduced during IgG purification, ii) human monocytes without mouse IgG to assess the nonspecific inhibitory effect of the monocytes, iii) immunized mouse. IgG without human monocytes to assess the direct inhibitory effect of antibodies, iv) purified IgG from hyperimmune African adults (Bouharoun-Tayoun, Attanath et al. 1990) as a positive control, and v) purified IgG from a pool of serum samples taken from the 30 animals before the first immunization as a negative control. After 48 and 72 hours of growth, respectively, 50 μl of RPMI supplemented with 0.5% albumax (GIBCO®), 100 U of penicillin and streptomycin per ml was added per well. After 96 hours of growth, parasitemia was determined by microscopic counting of more than 50,000 RBCs on Giemsa stained film. The specific growth inhibitory index (SGI) was calculated as follows (Soe, Singh et al. 2002):

100×{1-[(percent parasitemia with monocytes and test antibody/percent parasitemia with test antibody without monocytes)/(percent parasitemia with negative control antibody with monocytes/percent parasitemia with negative control antibody without monocytes)]}.

The direct inhibition of purified IgG (DI %) on parasite growth, i.e., in the absence of monocytes, was calculated as: ((parasitemia of culture with neither antibody nor monocyte)−(parasitemia of test IgG without monocyte))/(parasitemia of culture with neither antibody nor monocyte)×100.

2.A.10. RP-HPLC Analysis of GLURP and GLURP-MSP3

Samples were analyzed on a HPLC system (Phamacia®, Sweden), using a Protein C4 column (VYDAC®, 214TP54, USA) in a Acetonitrile:H₂O:TFA buffer system. Purified samples were diluted 1:2 in A-buffer (H₂O+0.1% (w/v) TFA), applied to the column, and bound material was eluted with a linear gradient (0-80%) of B-buffer (80% Acetonitrile+0.1% (w/v) TFA) over 20 column volumes. Elution was monitored by absorption at 214 nm. Peaks were collected, vacuum dried on a HetoVac (Heto, Denmark) and kept at 4° C. until use.

2.A.11. Maldi-Tof MS and Electrospray Mass Spectrometry

Samples for peptide mass mapping for were cut out of a coomassie stained SDS-PAGE gel. Half a band (approx. 1 μg protein) was washed, dried, reduced and alkylated with iodoacetamide before being digested overnight by modified trypsin (Promega, USA), essentially as described (Shevchenko, Wilm et al. 1996). The supernatant of the digest was applied to GELoader tips (Eppendorff®, Germany) packed with Poros 20 R2 reversed phase material (PerSeptive®, USA) and eluted with 0.8 μl of alpha-cyanohydroxycinnamic acid (20 μg/μl in 70% acetonitrile/30% water) directly onto the MALDI target (Kussmann, Lassing et al. 1997). Analysis was carried out on a PerSeptive Voyager STR (PerSeptive®, USA) operated in the reflector mode and the results were analyzed in GPMAW ver. 5.02 (Lighthouse data®, Denmark). Electrospray mass spectrometry (ES-MS) of the intact protein was carried out a Micromass QTOF (Micromass®, UK) using a nanospray source.

2.B. Results

2.B.1. Expression of Glurp and MSP3 in L. lactis

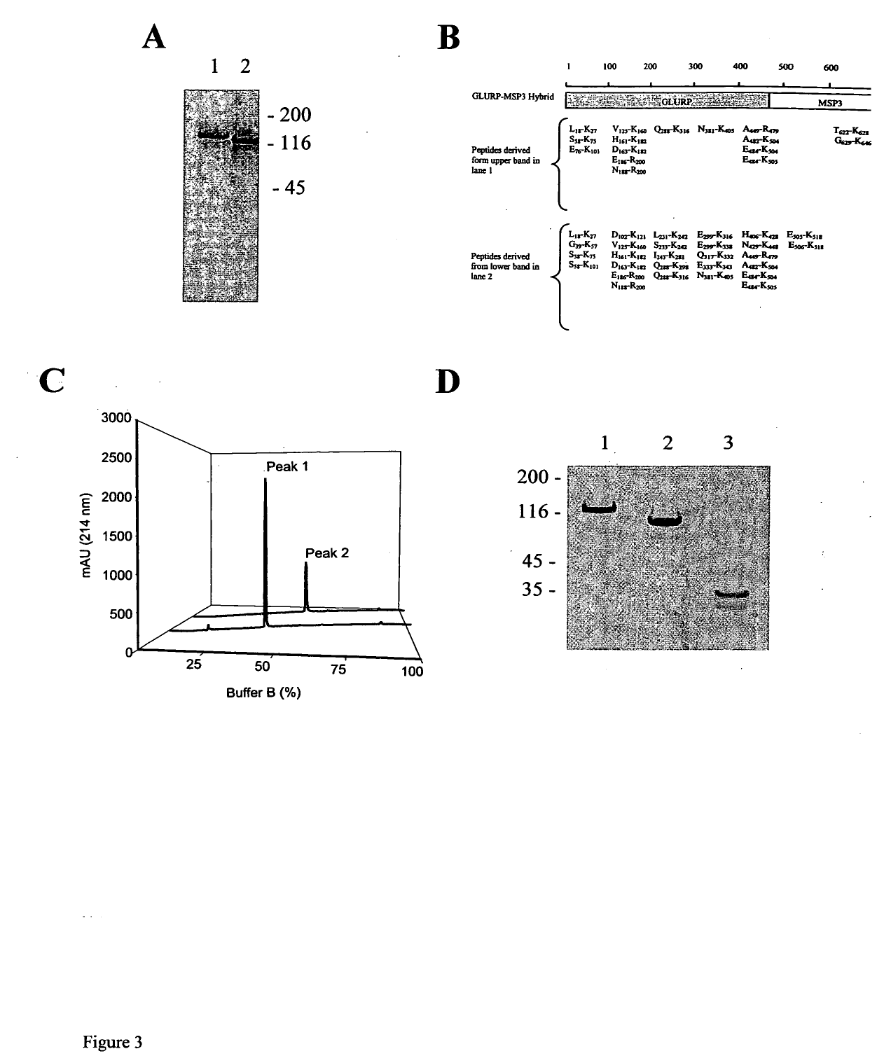

PCR fragments encoding the glurp₇₉₋₁₅₀₀ and MSP3₆₂₈₋₁₁₄₀ regions were cloned side by side thereby creating an in-frame fusion between a vector-encoded signal-peptide and a GLURP₂₇₋₅₀₀-MSP3₂₁₂₋₃₈₀ fusion protein (pKBR11, FIG. 2). This hybrid protein contains two additional amino acid residues created by joining these glurp and MSP3 fragments. The plasmid was transformed into L. lactis MG1363 and the resulting strain secreted the GLURP-MSP3 hybrid protein into the culture supernatant from where it was purified by sequential ion exchange on HiTrap Q and SP Sepharose columns followed by hydrophobic interaction chromatography on Phenyl Sepharose. Although these purification steps allowed us to remove most of the unwanted L. lactis proteins, it also became evident that the GLURP-MSP3 hybrid protein eluted as two distinct peaks from the Phenyl Sepharose column. Subsequent SDS-PAGE showed that these peaks contain protein band of approximately 140 and 143 kDa, respectively (FIG. 3A). When analyzed by immunoblotting both products were specifically recognized by antibodies to GLURP and MSP3 respectively, suggesting that the lower MW-band in lane 2 may result from incomplete translation of the mRNA and/or from protease cleavage of the primary protein product in lane 1. Both bands were excised for sequence identification using mass spectrometry. The sequence of a total of 16 tryptic fragments derived from the top band matched the GLURP and MSP3 sequences listed in FIG. 3B, showing that the recombinant protein corresponds to full-length GLURP₂₇₋₅₀₀-MSP3₂₁₂₋₃₈₀ fusion protein, whereas the sequences of 28 tryptic fragments from the bottom band suggests that this recombinant protein is derived from GLURP₂₇₋₅₀₀-MSP3₂₁₂₋₃₈₀ by proteolytic cleavage around residue 586 in the MSP3-region of the hybrid protein (FIG. 3B). The molecular mass of full-length GLURP₂₇₋₅₀₀-MSP3₂₁₂₋₃₈₀ was 74950 Da (±20 Da) as determined by ES-MS. Assuming that the recombinant protein contain the vector encoded amino acid residues A-E-R-S at the N-terminal end (FIG. 2), this molecular weight corresponds well to the predicted value of 74939. Thus, the GLURP-MSP3 hybrid protein in peak 2 is intact and contains the amino acid residues predicted from the nucleotide sequence. Subsequent reverse-phase chromatography further showed that the full-length (peak 2) and truncated (peak 1) hybrid proteins eluted with different retention-time from a C4 column (FIG. 3C).

For comparison, the individual glurp₇₃₋₁₅₄₂ and MSP3₆₂₈₋₁₁₄₀ fragments were also cloned (FIG. 2, pKBR8 and pKBR10) and the resulting recombinant proteins were purified to homogeneity by ion exchange (FIG. 3D, lanes 2 and 3).

2.B.2. Antigenicity of Recombinant GLURP and MSP3 Products

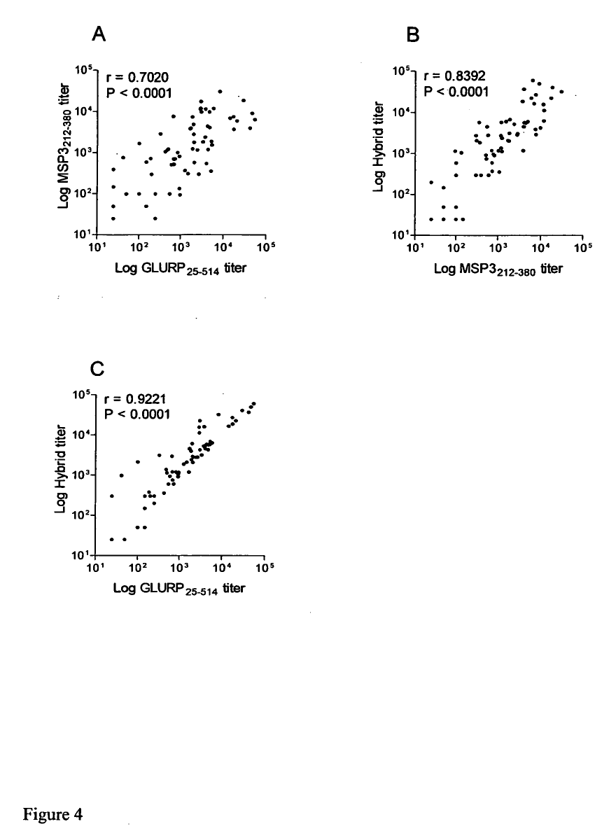

The antigenicity of the recombinant proteins was evaluated by ELISA against IgG antibodies from 71 adults Liberians clinically immune to malaria (FIG. 4). Serial dilutions of all plasma samples were tested on separate plates coated with each recombinant protein and the antigen-specific titer was determined as the dilution giving an absorbance of 1000. As expected, different plasma contained different amounts of GLURP and MSP3-specific IgG antibodies (FIG. 4A). In general, hybrid-specific antibody titers exceeded those recorded with the individual GLURP₂₅₋₅₁₄ and MSP3 antigens (FIGS. 4B and C) suggesting that the hybrid molecule provides an adequate presentation of GLURP and MSP3 antigenic determinants, respectively.

2.B.4. Immunogenicity of Recombinant GLURP and MSP3 Products

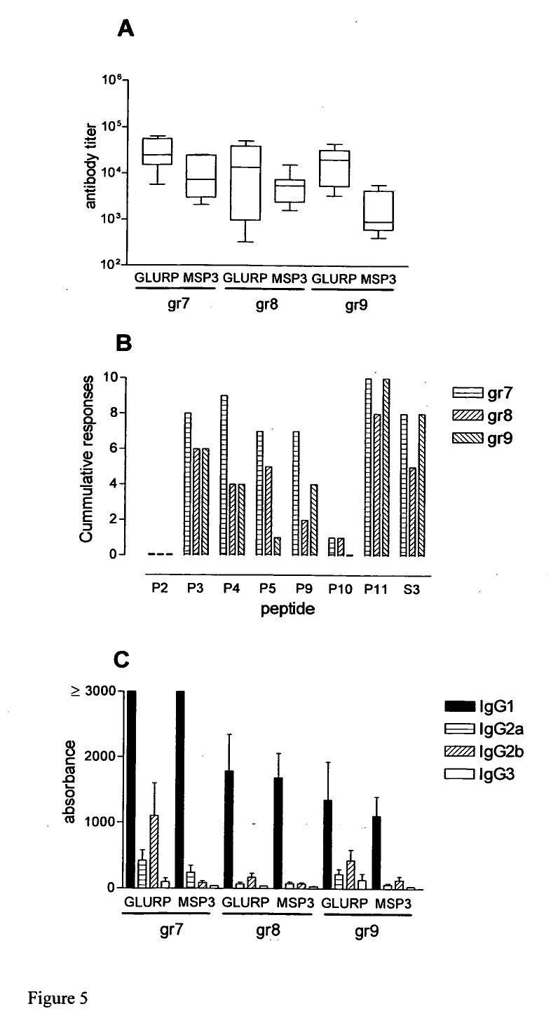

To determine whether the GLURP-MSP3 hybrid molecule is a superior immunogen compared to a mixture of the individual GLURP₂₅₋₅₁₄ and MSP3₂₁₂₋₃₈₀ molecules, groups of BALBc/CF1 mice were each immunized subcutaneously with the hybrid molecule in Montanide or with the individual GLURP₂₅₋₅₁₄ and MSP3₂₁₂₋₃₈₀ proteins combined in either one syringe or injected separately at two different sites. Sera collected 35 days after the first injection, were tested for IgG antibody reactivity against GLURP and MSP3, respectively. While the mean GLURP-ELISA titer is only marginally higher in the hybrid group than in the other two groups, mean MSP3-ELISA titer is 4.3-fold higher (Kruskal Wallis test, P<0.004) in the group receiving the hybrid compared to the group receiving both MSP3₂₁₂₋₃₈₀ and GLURP₂₅₋₅₁₄ at two different sites (compare gr7 and gr9 in FIG. 5A). At the individual level, mice immunized with the hybrid reacted strongly with both GLURP and MSP3 domains whereas mice immunized with a combination of two molecules tended to mount a predominant antibody response against either GLURP or MSP3. The anti-hybrid IgG antibodies are mainly directed against the GLURP-derived P3, P4, P11, and S3 peptides containing known epitopes for human antibodies (Theisen, Soe et al. 2000); however peptides P5 and P9 which do not contain such epitopes were also recognized (FIG. 5B). Whereas the GLURP and MSP3-specific IgG subclass profiles are similar for all vaccine formulations (FIG. 5C), GLURP-specific IgG antibodies use preferentially the Kappa light chain and MSP3-specific IgG antibodies preferentially the Lambda light chain. This difference in light chain was found for all GLURP or MSP3-specific antibodies whether raised against the hybrid or the mixtures of the individual molecules.

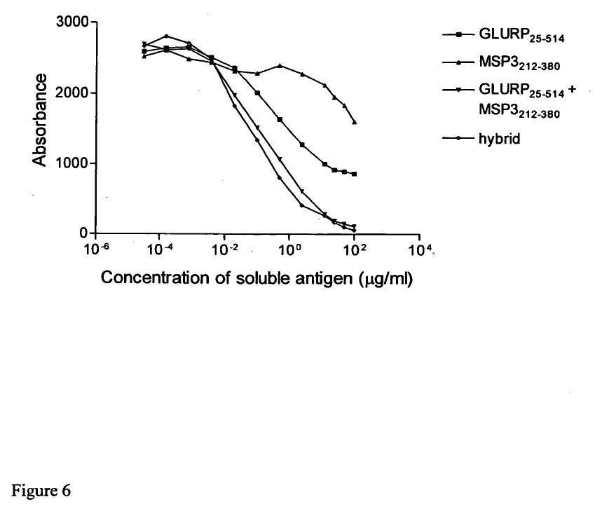

The specificity of mouse antibodies to the hybrid was also analyzed by competition-ELISA (FIG. 6). It appears that antibodies to the hybrid are purely GLURP and MSP3-specific, since a mixture of soluble GLURP₂₅₋₅₁₄ and MSP3₂₁₂₋₃₈₀ could completely inhibit the binding of anti-hybrid antibodies to immobilized GLURP₂₇₋₅₀₀-MSP3₂₁₂₋₃₈₀. Thus, the construction of a GLURP-MSP3 hybrid molecule has not led to create new B-cell epitopes in the overlapping area.

2.B.5. Reactivity of Mouse Anti-GLURP and Anti-MSP3 Sera with Native GLURP and MSP3

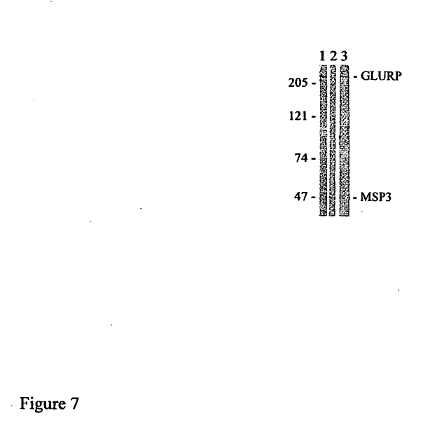

The immunogenicity of the recombinant GLURP and MSP3 was also investigated by immunoblotting of parasite-derived proteins with sera from mice immunized with each of the three recombinant proteins, hybrid, GLURP₂₅₋₅₁₄ and MSP3₂₁₂₋₃₈₀, respectively. As demonstrated in FIG. 7, plasma from mice immunized with GLURP₂₅₋₅₁₄, MSP3₂₁₂₋₃₈₀, and the hybrid recognized polypeptides of approximately 220,000 Da (lane 1), 48,000 Da (lane 2), and both (lane 3), respectively.

2.B.6. Biological Effect of the Hybrid-Induced Antibodies

Since affinity-purified human antibodies to GLURP and MSP3 can inhibit parasite growth in vitro in cooperation with human monocytes (Oeuvray, Bouharoun-Tayoun et al. 1994; Theisen, Soe et al. 1998; Theisen, Soe et al. 2000) it was investigated whether anti-hybrid mouse antibodies had a similar effect. Total IgG was isolated from groups of mice, which had received the hybrid (gr7), or the individual GLURP₂₅₋₅₁₄ and MSP3₂₁₂₋₃₈₀ molecules respectively. The three IgG solutions were adjusted to the same IFA end-point, i.e. the same degree of reactivity to parasite proteins to provide comparative data, and their effect on parasite growth was determined. The three IgG suspensions all exerted a strong inhibitory effect in the presence of normal human monocytes whereas they only had a non-significant effect in the absence of monocytes (Table 3). The pre-bleed did not promote inhibition of parasite growth indicating that the observed ADCI-effects are due to the presence of specific antibodies to GLURP and MSP3 (Table 3). There was no correlation between inhibitory effect and the IgG concentration (Table 3).

TABLE 1
Table 1. Specific growth inhibition index (SGI) and direct growth
inhibition (DI) of total IgG purified from serum pools of mice
immunized with GLURP₂₇₋₅₀₀-MSP3₂₁₂₋₃₈₀ or
the individual GLURP₂₅₋₅₁₄ and MSP3₂₁₂₋₃₈₀ molecules,
respectively. The unspecific growth inhibitory effect of mouse IgG was
measured with total IgG purified from the pooled pre-bleeds of all animals
for comparison. Each IgG was tested in three different concentrations
adjusted according to their IFA titer. The corresponding IgG concentration
is indicated for each assay. SGI and DI values are the results of three
independent assays.

| IgG from mice | IFA titer | IgG concentratio [symbol] | SGI (%) ± | DI |
|---|---|---|---|---|
| immunized with | in assay | assay (μg/ml) | SD | (%) ± SD |
| hybrid | 30 | 87 | 58 ± 3 | 0 |
| | 20 | 58 | 64 ± 5 | 0 |
| | 10 | 29 | 63 ± 10 | 0 |
| GLURP | 10 | 110 | 63 ± 5 | 0 |
| | 5 | 55 | 37 ± 3 | 0 |
| MSP3 | 10 | 110 | 57 ± 4 | 0 |
| | 5 | 55 | 58 ± 5 | 0 |
| pre-bleed | <5 | 110 | 20 ± 10 | 10 ± 5 |
| | 0 | 55 | 8 ± 3 | 10 ± 5 |

2.C. Discussion

Lactic acid bacteria have a long history of use in the production of fermented foods. However, their use in the pharmaceutical field as production hosts for recombinant proteins or as live delivery vehicles for administration of vaccines has only been initiated recently. The testing of new vaccines and therapeutics continuously poses a challenge to the development of suitable gene expression systems for heterologous protein production. The L. lactis P170 based gene expression systems employed here has proved” ideal for small-scale production of malaria antigens because: i) production yields are acceptable, as compared to other vectors ii) the products are biologically active without undesired modifications iii) the fermentation process is well established iv) the recombinant products are secreted and v) it offers a simple purification process with little protein degradation. The GLURP and MSP3 molecules have previously been expressed separately in E. coli with and without various affinity-tags (McColl, Silva et al. 1994; Theisen, Cox et al. 1994; Theisen, Vuust et al. 1995; Theisen, Thomas et al. 2001), however, these products were considered far less suitable for testing in human beings because host immune responses against the affinity-tag could occur and impede repeated immunizations. In addition, at least GLURP-R0 was highly unstable when produced without a fusion partner in E. coli (unpublished). The inventors have noted that the His-tag can also change the physical properties of the recombinant protein, making it less soluble in aqueous solution than the untagged version.

In the present study, a chimeric malaria protein containing the 5′-end of P. falciparum glurp fused in frame to the 3′-end of P. falciparum MSP3 as well as the individual GLURP and MSP3 domains was constructed in order to identify the main antigenic determinants on the hybrid protein. Each of these constructs gave rise to a major dominant full-length product and a lower-molecular mass band, which corresponds to degradation products. L. lactis contains a surface associated housekeeping protease, HtrA, which might be responsible for these smaller molecular-mass products. Inactivation of the htrA gene leads to stabilization of several recombinant proteins produced and secreted by L. lactis (Poquet, Saint et al. 2000; Miyoshi, Poquet et al. 2002). This might also be the case in this study and further investigations should include expression studies of the malaria antigens in an, htrA mutant strain.

In L. lactis the nascent recombinant proteins should be cleaved by leader peptidase I since they contain the predicted cleavage sequence Gln-Ala_Ala-Glu (http://www.cbs.dtu.dk/services/SignalP/) thereby leaving; the Ala-Glu amino acid residues attached to the N-terminal end of the mature protein. This prediction was confirmed by the excellent agreement between the theoretical and experimental molecular weights of the hybrid (74939 vs. 74950 Da±20 Da). In addition to the vector-encoded amino acids at the N-terminal end, the hybrid also contains two residues (Arg-Ser) in the fusion junction between GLURP and MSP3. These amino acids however, did not form part of a novel B-cell epitope in the overlapping area since anti-hybrid antibodies generated in outbreed mice were exclusively directed against antigenic determinants in the respective GLURP and MSP3 regions.

The immunogenicity of the hybrid was studied in mice with Montanide used as the adjuvant since Montanide was used in recent clinical trials with long synthetic peptides derived, from GLURP and MSP3, respectively. Immunizations with the protein-hybrid consistently generated a stronger antibody response against the individual GLURP and MSP3 domains than any other combination of the two molecules, thereby validating the value of the approach. The difference was most pronounced for the MSP3-specific antibody response suggesting that T cell epitopes located in the GLURP region provide help for B-cell epitopes in the MSP3 region. When the animals were injected with a mixture of the two molecules in one syringe, individual mice tended to mount a predominant antibody response against either GLURP or MSP3. In some animals GLURP was immuno-dominant whereas in other animals it was MSP3. This phenomenon was not observed, when GLURP was injected in one part and MSP3 in another part of the body suggesting that the concomitant presentation of a mixture of individual GLURP and MSP3 proteins to the same antigen-presenting cells may lead to competition. Whichever the underlying mechanism, the present data support the conclusion that the hybrid molecule provides a superior presentation of GLURP and MSP3 antigenic determinants compared to the individual molecules. This conclusion is also in agreement with the observation that the hybrid was more effectively recognized by naturally occurring IgG antibodies in clinically immune African adults than the individual antigens.

Antibodies raised against the hybrid reacted strongly with native parasite proteins by IFA and recognized parasite-expressed GLURP and MSP3 by immunoblotting analysis. The hybrid-specific antibodies did not inhibit parasite growth alone, however, when allowed to collaborate with human monocytes they proved strongly inhibitory in several independent ADCI assays. While there are many examples of mouse antibodies which can interfere with merozoite invasion of red blood cells (Thomas, Deans et al. 1984; Pirson and Perkins 1985; Berzins, Perlmann et al. 1986; Epping, Goldstone et al. 1988; Clark, Donachie et al. 1989; Blackman, Heidrich et al. 1990), this study is the first report of a mouse IgG preparation, which act synergistically with human monocytes to inhibit P. falciparum parasite growth in vitro. The human FcγIIa receptor is believed to be the primary trigger molecule involved in ADCI (Bouharoun-Tayoun, Oeuvray et al. 1995. This receptor exists in two different alloforms which are known to be polymorphic with respect to the interaction with mouse IgG1 (Parren, Warmerdam et al. 1992). The GLURP and MSP3 specific mouse antibodies were mainly of the IgG1 and IgG2b isotypes, two antibodies which bind well to the most prevalent FcγIIa receptor with arginine at position 131 (Pleass and Woof 2001). This provides pre-clinical indications that hybrid-specific antibodies generated by immunization mimic the ADCI-effect of naturally occurring human antibodies against GLURP (Theisen, Soe et al. 1998) and MSP3 (Oeuvray, Bouharoun-Tayoun et al. 1994), respectively.

The value of the hybrid strategy in humans was recently strengthened by immunoepidemiological studies in an Asian setting: besides confirming the strong statistical association between protection and both anti-GLURP and anti-MSP3 antibody responses, it further showed that in those rare individuals not responding to either antigen, a response to the other antigen was always present and associated with protection. Hence, when presented by the parasite, the antigenicity of the two molecules constituting the hybrid is complementary.

The present example hence discloses a GLURP-MSP3 hybrid molecule, which i) is suitable for use in human clinical trials, ii) is more immunogenic in experimental models than the individual GLURP and MSP3 domains, and iii) can induce specific antibodies in mice which inhibit parasite growth in collaboration with human monocytes.

### Example 3

### In Vivo Passive Transfer Experiments of Anti-LURP and Anti-MSP3 Antibodies in an Immunocompromised Mouse Model

Using the P. falciparum Hu RBC BXN immunocompromised mouse model (Badell, E., C. Oeuvray, et al., 2000), the effect of several human affinity purified antibodies was studied in passive transfer experiments in vivo.

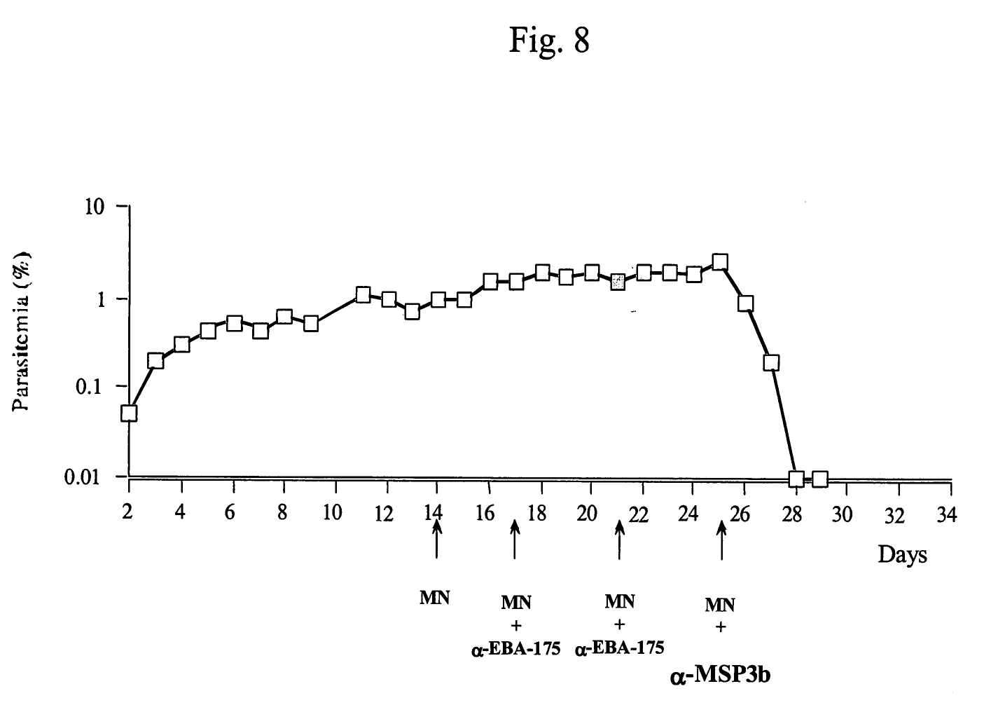

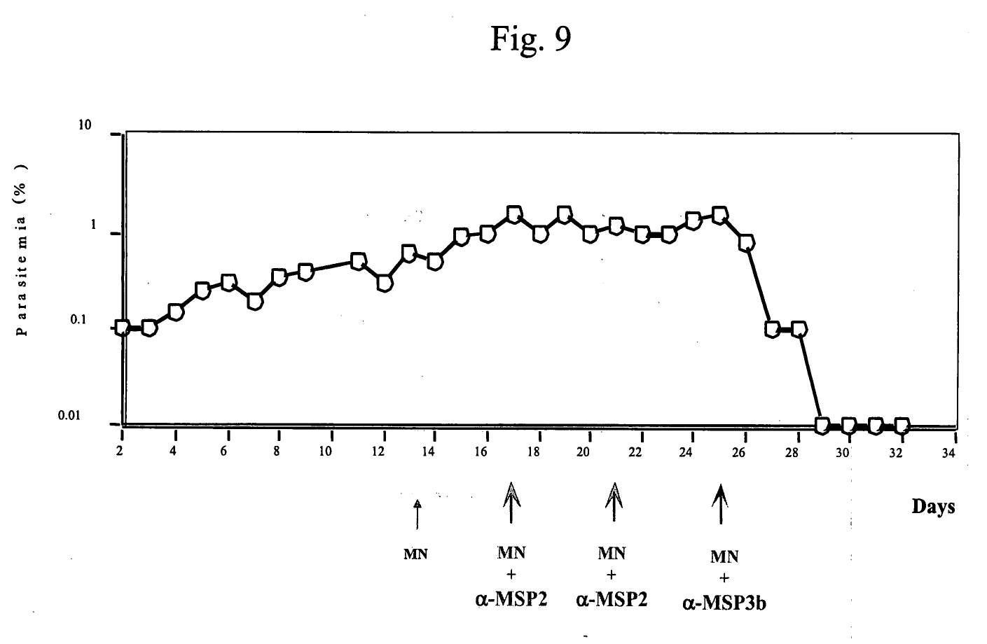

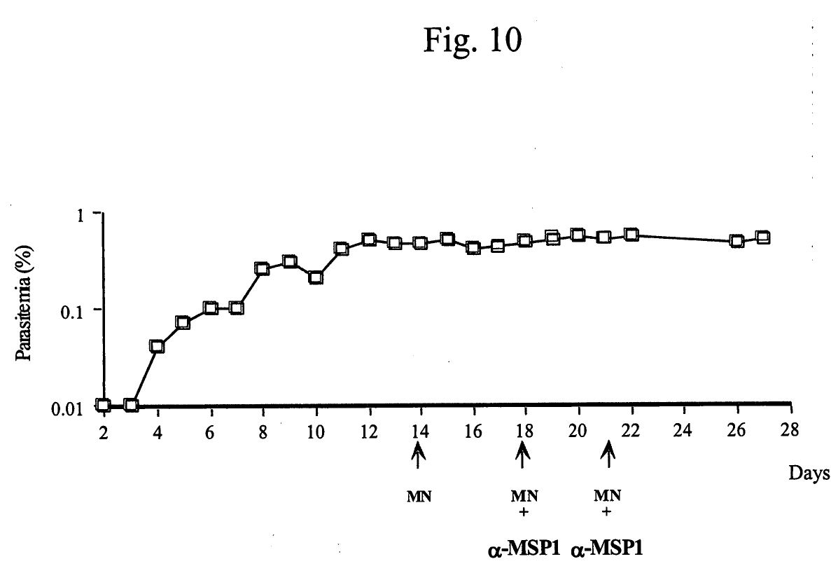

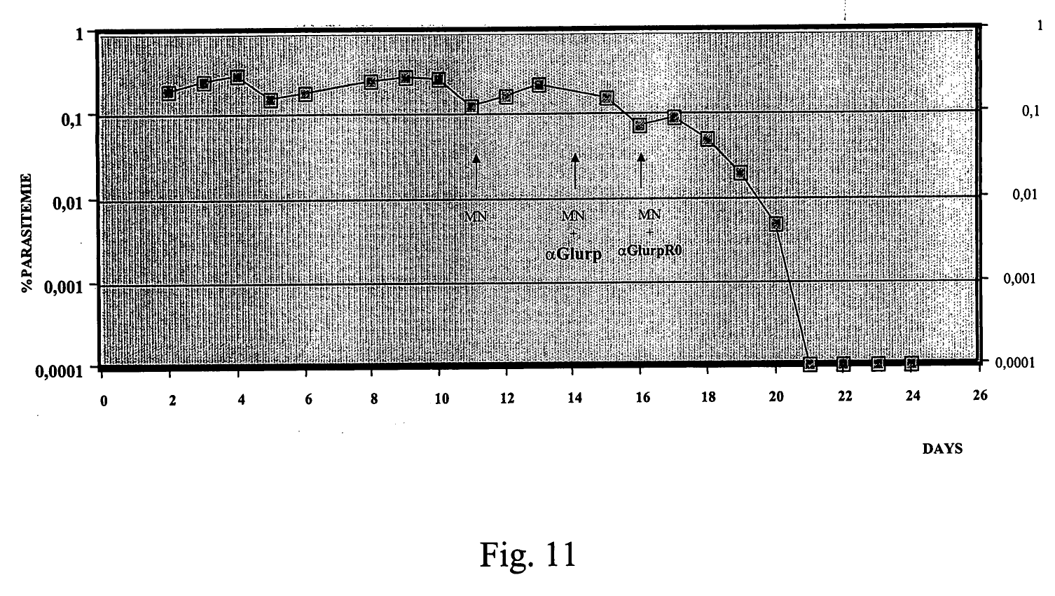

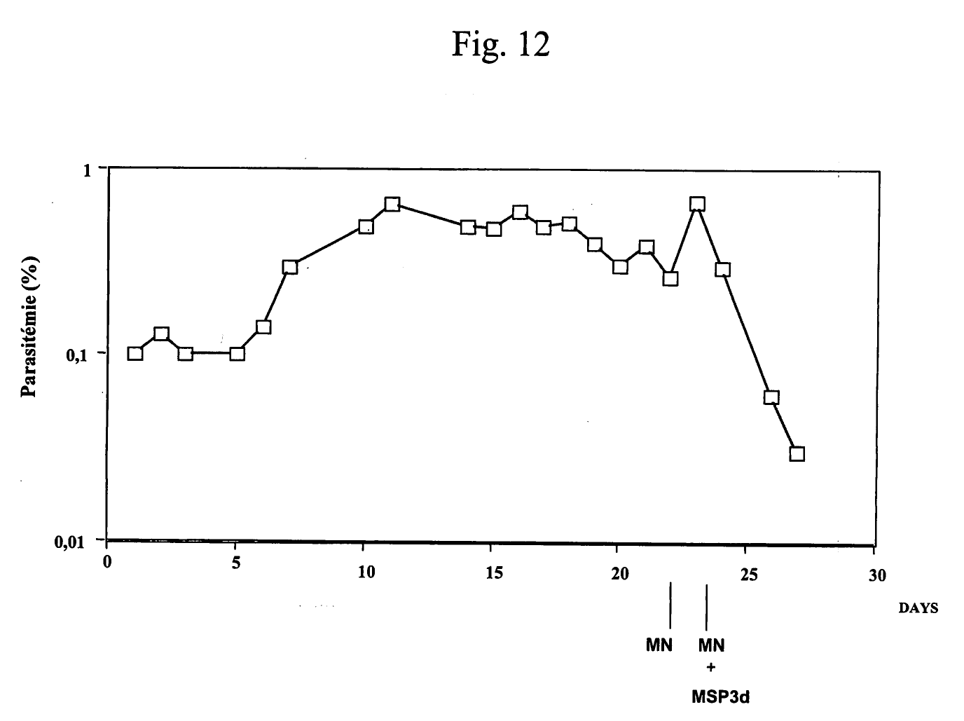

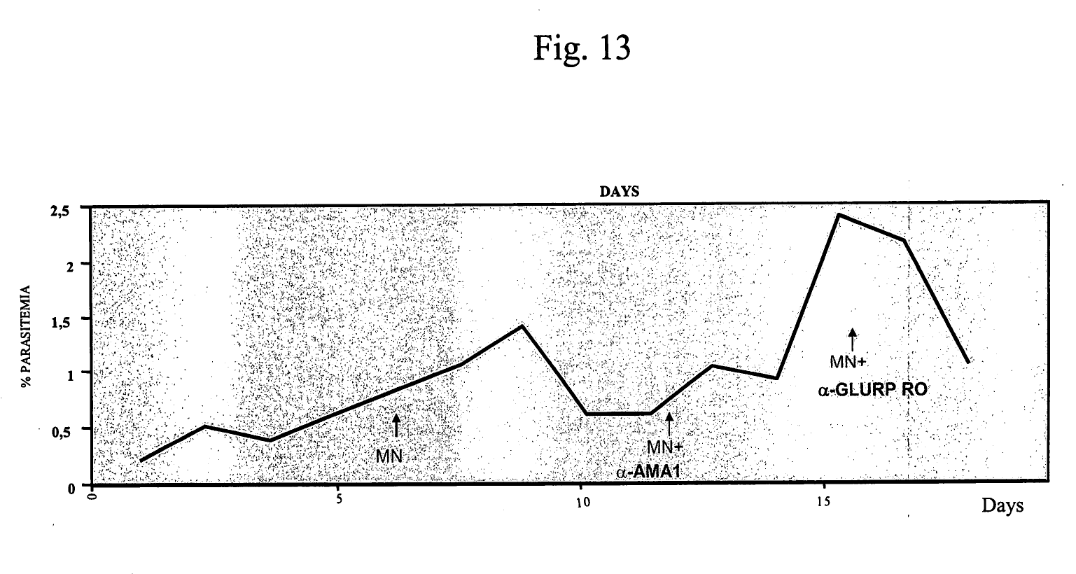

As shown in FIGS. 8 to 12, anti-GLURP antibodies were effective at clearing the P. falciparum parasitemia (FIG. 11) whereas, in the same animal, antibodies to EBA-175 (FIG. 8), MSP2 (FIG. 9), and MSP1 (FIG. 10) had no beneficial effect, and antibodies to the AMA1 vaccine candidate had the opposite effect, i.e. increased the parasitemia (FIG. 13). However, the effect of anti-GLURP antibodies is slower than that of anti-MSP3 antibodies clearance of parasitaemia in 4-5 days (FIG. 11) compared to 2-3 days, respectively (FIGS. 8, 9 and 12).

This study confirms, under in vivo conditions, by passive transfer of antibodies elicited in humans, the indications obtained under in vitro conditions in the ADCI assay. Since the results obtained by in vitro methods are coherent with the results obtained under in vivo conditions in the BXN mouse model, these results concur to confirm the validity of a combined malaria vaccine associating GLURP and MSP-3, since both antibodies have a profound effect on P. falciparum.

### REFERENCES

Badell, E., C. Oeuvray, et al. (2000). “Human malaria in immunocompromised mice: an in vivo model to study defense mechanisms against Plasmodium falciparum.” J Exp Med 192(11): 1653-60.
Beier, J. C., G. F. Killeen, et al. (1999). “Short report: entomologic inoculation rates and Plasmodium falciparum malaria prevalence in Africa.” Am J Trop Med Hyg 61(1): 109-13.
Berzins, K., H. Perlmann, et al. (1986). “Rabbit and human antibodies to a repeated amino acid sequence of a Plasmodium falciparum antigen, Pf 155, react with the native protein and inhibit merozoite invasion.”Proc Natl Acad Sci USA 83(4): 1065-9.
Blackman, M. J., H. G. Heidrich, et al. (1990). “A single fragment of a malaria merozoite surface protein remains on the parasite during red cell invasion and is the target of invasion-inhibiting antibodies.” J Exp Med 172(1): 379-82.
Bottius, E., L. BenMohamed, et al. (1996). “A novel Plasmodium falciparum sporozoite and liver stage antigen (SALSA) defines major B, T helper, and CTL epitopes.” J Immunol 156(8): 2874-84.
Bouharoun-Tayoun, H., P. Attanath, et al. (1990). “Antibodies that protect humans against Plasmodium falciparum blood stages do not on their own inhibit parasite growth and invasion in vitro, but act in cooperation with monocytes.” J Exp Med 172(6): 1633-41.
Bouharoun-Tayoun, H. and P. Druilhe (1992). “Antibodies in falciparum malaria: what matters most, quantity or quality?” Mem Inst Oswaldo Cruz 87 Suppl 3: 229-34.
Bouharoun-Tayoun, H., C. Oeuvray, et al. (1995). “Mechanisms underlying the monocyte-mediated antibody-dependent killing of Plasmodium falciparum asexual blood stages.” J Exp Med 182(2): 409-18.
Clark, J. T., S. Donachie, et al. (1989). “46-53 kilodalton glycoprotein from the surface of Plasmodium falciparum merozoites.” Mol Biochem Parasitol 32(1): 15-24.
Dame, J. B., J. L. Williams, et al. (1984). “Structure of the gene encoding the immunodominant surface antigen on the sporozoite of the human malaria parasite Plasmodium falciparum.” Science 225(4662): 593-9.
Daubersies, P., A. W. Thomas, et al. (2000). “Protection against Plasmodium falciparum malaria in chimpanzees by immunization with the conserved pre-erythrocytic liver-stage antigen 3.” Nat Med 6(11): 1258-63.
de Stricker, K., J. Vuust, et al. (2000). “Conservation and heterogeneity of the glutamate-rich protein (GLURP) among field isolates and laboratory lines of Plasmodium falciparum.” Mol Biochem Parasitol 111(1): 123-30.
Dodoo, D., T. G. Theander, et al. (1999). “Levels of antibody to conserved parts of Plasmodium falciparum merozoite surface protein 1 in Ghanaian children are not associated with protection from clinical malaria.” Infect Immun 67(5): 2131-7.
Dodoo, D., M. Theisen, et al. (2000). “Naturally acquired antibodies to the glutamate-rich protein are associated with protection against Plasmodium falciparum malaria.” J Infect Dis 181(3): 1202-5.
Egan, A. F., J. Morris, et al. (1996). “Clinical immunity to Plasmodium falciparum malaria is associated with serum antibodies to the 19-kDa C-terminal fragment of the merozoite surface antigen, PfMSP-1.” J Infect Dis 173(3): 765-9.
Epping, R. J., S. D. Goldstone, et al. (1988). “An epitope recognised by inhibitory monoclonal antibodies that react with a 51 kilodalton merozoite surface antigen in Plasmodium falciparum.” Mol Biochem Parasitol 28(1): 1-10.
Escalante, A. A., H. M. Grebert, et al. (2001). “Polymorphism in the gene encoding the apical membrane antigen-1 (AMA-1) of Plasmodium falciparum. X. Asembo Bay Cohort Project.” Mol Biochem Parasitol 113(2): 279-87.
Fidock, D. A., E. Bottius, et al. (1994). “Cloning and characterization of a novel Plasmodium falciparum sporozoite surface antigen, STARP.” Mol Biochem Parasitol 64(2): 219-32.
Gasson, M. J. (1983). “Plasmid complements of Streptococcus lactis NCDO 712 and other lactic streptococci after protoplast-induced curing.” J. Bacteriol 154(1): 1-9.
Guerin-Marchand, C., P. Druilhe, et al. (1987). “A liver-stage-specific antigen of Plasmodium falciparum characterized by gene cloning.” Nature 329(6135): 164-7.
Holo, H. and I. F. Nes (1995). “Transformation of Lactococcus by electroporation.” Methods Mol Biol 47: 195-9.
Huber, W., I. Felger, et al. (1997). “Limited sequence polymorphism in the Plasmodium falciparum merozoite surface protein 3.” Mol Biochem Parasitol 87(2): 231-4.
Israelsen, H., S. M. Madsen, et al. (1995). “Cloning and partial characterization of regulated promoters from Lactococcus lactis Tn917-lacZ integrants with the new promoter probe vector, pAK80.” Appl Environ Microbiol 61(7): 2540-7.
Kang, Y., P. A. Calvo, et al. (1998). “Comparison of humoral immune responses elicited by DNA and protein vaccines based on merozoite surface protein-1 from Plasmodium yoelii, a rodent malaria parasite.” J Immunol 161(8): 4211-9.
Khusmith, S. and P. Druilhe (1983). “Cooperation between antibodies and monocytes that inhibit in vitro proliferation of Plasmodium falciparum.” Infect Immun 41(1)-219-23.
Klausen, J., M. Magnusson, et al. (1994). “Characterization of purified protein derivative of tuberculin by use of monoclonal antibodies: isolation of a delayed-type hypersensitivity reactive component from M. tuberculosis culture filtrate.” Scand J Immunol 40(3): 345-9.
Knapp, B., E. Hundt, et al. (1989). “Molecular cloning, genomic structure and localization in a blood stage antigen of Plasmodium falciparum characterized by a serine stretch.” Mol Biochem Parasitol 32(1): 73-83.
Kussmann, M., U. Lassing, et al. (1997). “Matrix-assisted laser desorption/ionization mass spectrometric peptide mapping of the neural cell adhesion protein neurolin purified by sodium dodecyl sulfate polyacrylamide gel electrophoresis or acidic precipitation.” J Mass Spectrom 32(5): 483-93.
Lunel, F. and P. Druilhe (1989). “Effector cells involved in nonspecific and antibody-dependent mechanisms directed against Plasmodium falciparum blood stages in vitro.” Infect Immun 57(7): 2043-9.
Madsen, S. M., J. Arnau, et al. (1999). “Molecular characterization of the pH-inducible and growth phase-dependent promoter P170 of Lactococcus lactis.” Mol Microbiol 32(1): 75-87.
Marshall, V. M., W. Tieqiao, et al. (1998). “Close linkage of three merozoite surface protein genes on chromosome 2 of Plasmodium falciparum.” Mol Biochem Parasitol 94(1): 13-25.
McColl, D. J. and R. F. Anders (1997). “Conservation of structural motifs and antigenic diversity in the Plasmodium falciparum merozoite surface protein-3 (MSP-3).” Mol Biochem Parasitol 90(1): 21-31.
McColl, D. J., A. Silva, et al. (1994). “Molecular variation in a novel polymorphic antigen associated with Plasmodium falciparum merozoites.” Mol Biochem Parasitol 68(1): 53-67.
Miller, L. H., T. Roberts, et al. (1993). “Analysis of sequence diversity in the Plasmodium falciparum merozoite surface protein-1 (MSP-1).” Mol Biochem Parasitol 59(1): 1-14.
Miyoshi, A., I. Poquet, et al. (2002). “Controlled production of stable heterologous proteins in Lactococcus lactis.” Appl Environ Microbiol 68(6): 3141-6.
Oeuvray, C., H. Bouharoun-Tayoun, et al. (1994). “Merozoite surface protein-3: a malaria protein inducing antibodies that promote Plasmodium falciparum killing by cooperation with blood monocytes.” Blood 84(5): 1594-602.
Oeuvray, C., M. Theisen, et al. (2000). “Cytophilic immunoglobulin responses to Plasmodium falciparum glutamate-rich protein are correlated with protection against clinical malaria in Dielmo, Senegal.” Infect Immun 68(5): 2617-20.
Parren, P. W., P. A Warmerdam, et al. (1992). “On the interaction of IgG subclasses with the low affinity Fc gamma RIIa (CD32) on human monocytes, neutrophils, and platelets. Analysis of a functional polymorphism to human IgG2.” J Clin Invest 90(4): 1537-46.
Peterson, M. G., V. M. Marshall, et al. (1989). “Integral membrane protein located in the apical complex of Plasmodium falciparum.” Mol Cell Biol 9(7): 3151-4.
Pirson, P. J. and M. E. Perkins (1985). “Characterization with monoclonal antibodies of a surface antigen of Plasmodium falciparum merozoites.” J Immunol 134(3): 1946-51.
Pleass, R. J. and J. M. Woof (2001). “Fc receptors and immunity to parasites.” Trends Parasitol 17(11): 545-51.
Poquet, I., V. Saint, et al. (2000). “HtrA is the unique surface housekeeping protease in Lactococcus lactis and is required for natural protein processing.” Mol Microbiol 35(5): 1042-51.
Robson, K. J., J. R. Hall, et al. (1988). “A highly conserved amino-acid sequence in thrombospondin, properdin and in proteins from sporozoites and blood stages of a human malaria parasite.” Nature
Shevchenko, A., M. Wilm, et al. (1996). “Mass spectrometric sequencing of proteins silver-stained polyacrylamide gels.” Anal Chem 68(5): 850-8.
Simmons, D., G. Woolleft, et al. (1987). “A malaria protein exported into a new compartment within the host erythrocyte.” Embo J 6(2): 485-91.
Simon, D. and A. Chopin (1988). “Construction of a vector plasmid family and its use for molecular cloning in Streptococcus lactis.” Biochimie 70(4): 559-66.
Soe, S., A. Khin Saw, et al. (2001). “Premunition against Plasmodium falciparum in a malaria hyperendemic village in Myanmar.” Trans R Soc Trop Med Hyg 95(1): 81-4.
Soe, S., S. Singh, et al. (2002). “Plasmodium falciparum serine repeat protein, a new target of monocyte-dependent antibody-mediated parasite killing.” Infect Immun 70(12): 7182-4.
Theisen, M., G. Cox, et al. (1994). “Immunogenicity of the Plasmodium falciparum glutamate-rich protein expressed by vaccinia virus.” Infect Immun 62(8): 3270-5.
Theisen, M.; D. Dodoo, et al. (2001). “Selection of glutamate-rich protein long synthetic peptides for vaccine development: antigenicity and relationship with clinical protection and immunogenicity.” Infect Immun 69(9): 5223-9.
Theisen, M., S. Soe, et al. (2000). “Identification of a major B-cell epitope of the Plasmodium falciparum glutamate-rich protein (GLURP), targeted by human antibodies mediating parasite killing.” Vaccine 19(2-3): 204-12.
Theisen, M., S. Soe, et al. (1998). “The glutamate-rich protein (GLURP) of Plasmodium falciparum is a target for antibody-dependent monocyte-mediated inhibition of parasite growth in vitro.” Infect Immun 66(1): 11-7.
Theisen, M., A. W. Thomas, et al. (2001). “Cloning, nucleotide sequencing and analysis of the gene encoding the glutamate-rich protein (GLURP) from Plasmdium reichenowi.” Mol Biochem Parasitol 115(2): 269-73.
Theisen, M., J. Vuust, et al. (1995). “Antigenicity and immunogenicity of recombinant glutamate-rich protein of Plasmodium falciparum expressed in Escherichia coli.” Clin Diagn Lab Immunol 2(1): 30-4.
Thomas, A. W., D. A. Carr, et al. (1990). “Sequence comparison of allelic forms of the Plasmodium falciparum merozoite surface antigen MSA2.” Mol Biochem Parasitol 43(2): 211-20.
Thomas, A. W., J. A. Deans, et al. (1984). “The Fab fragments of monoclonal IgG to a merozoite surface antigen inhibit Plasmodium knowlesi invasion of erythrocytes.” Mol Biochem Parasitol 13(2): 187-99.
Tun-Lin, W., M. M. Thu, et al. (1995). “Hyperendemic malaria in a forested, hilly Myanmar village.” J Am Mosa Control Assoc 11 (4): 401-7.
Valenzuela, J. G., Y. Belkaid, et al. (2001). “Toward a defined anti-Leishmania vaccine targeting vector antigens: characterization of a protective salivary protein.” J Exp Med 194(3): 331-42.
Warmerdam, P. A., J. G. van de Winkel, et al. (1991). “A single amino acid in the second Ig-like domain of the human Fc gamma receptor II is critical for human IgG2 binding.” J Immunol 147(4): 1338-43.

1. A chimeric molecule comprising a GLURP moiety consisting of a polypeptide fragment of at least 50 amino acids from the GLURP₂₅₋₅₁₄ fragment of SEQ ID No:1, and a MSP3 moiety consisting of a polypeptide fragment of at least 50 amino acids from the MSP3₂₁₂₋₃₈₀ fragment of SEQ ID No:2, or a variant thereof in which 1 to 15 amino acids in any or both of said moieties have been deleted, added or changed by conservative substitution, wherein said chimeric molecule raises antibodies against both the polypeptides of SEQ ID No:1 and SEQ ID No:2, in mice immunized with it.

2-17. (canceled)
